SEQUENCE LISTING (8 sequences)

SEQ ID NO: 1 - DNA, 23 bp, Artificial
  forward primer
ggagctagaacgattcgcagtta

SEQ ID NO: 2 - DNA, 19 bp, Artificial
  reverse primer
cagctgctgcttgctgtgc

SEQ ID NO: 3 - DNA, 25 bp, Artificial
  qPCR reverse primer
ggttgtagctgtcccagtatttgtc

SEQ ID NO: 4 - DNA, 30 bp, Artificial
  FAM probe
acagccttctgatgtctctaaaaggccagg

SEQ ID NO: 5 - DNA, 23 bp, Artificial
  reverse primer
gtggtctgaattgggttatgagg

SEQ ID NO: 6 - DNA, 20 bp, Artificial
  forward primer
ggagggaagctcatcagtgg

SEQ ID NO: 7 - DNA, 21 bp, Artificial
  qPCR forward primer
ggagcttggaacagactcacg

SEQ ID NO: 8 - DNA, 29 bp, Artificial
  VIC Probe
acctcacctcagccattgaactcacttcg

(19) United States
(12) Patent Application Publication
(10) Pub. No.: US 2022/0325336 A1
(43) Pub. Date: Oct. 13, 2022

(54) TRANSDUCTION EFFICIENCY ASSAY

(71) Applicant: bluebird bio, Inc., Cambridge, MA (US)

(72) Inventor: Ilya Shestopalov, Billerica, MA (US)

(21) Appl. No.: 17/641,059

(22) PCT Filed: Sep. 7, 2020
(86) PCT No.: PCT/US2020/049627

§ 371 Date: Mar. 7, 2022

Related U.S. Application Data
(60) Provisional application No. 62/896,376, filed on Sep. 5, 2019.

(51) Int. Cl. C12Q 1/6851; C12Q 1/70; C12N 5/0783; C12N 15/62; C12N 15/86

(52) U.S. Cl. CPC C12Q 1/6851; C12Q 1/701; C12N 5/0636; C12N 15/625; C12N 15/86; C12N 2740/15043

(57) ABSTRACT

Disclosed herein are lentiviral vector transduction efficiency assays for gene therapy treatments. Also disclosed herein are methods for measuring transduction efficiency of a lentiviral vector.

### RELATED APPLICATIONS

This application claims the benefit of U.S. Provisional Application Ser. No. 62/896,376, filed Sep. 5, 2019, the contents of which is hereby incorporated by reference in its entirety.

### BACKGROUND OF THE INVENTION

The success of cell-based gene therapy relies on, in part, obtaining a sufficient number of vector-transduced cells in the final drug product (DP). Historical assessments of transduction efficiency have relied on calculating an average vector copy number (VCN) normalized across the population of cells in the drug product (expressed as VCN per diploid genome). However, VCN itself does not directly inform the percentage of cells containing at least one transgene. Moreover, the percentage of transduced, or lentiviral vector (LVV)-positive cells (% LVV+) may be an important attribute of certain gene therapy drug products—particularly for those in which the transgene functions cell autonomously. Measurement of % LVV+ cells can be complicated by the lack of expression of the transgenic protein in the assayed cells, absence of fluorescent reporters in clinical vectors, and/or lack of suitable methods for detection of transgene expression. Thus, there exists a need for improved methods for measuring the quantification of % LVV+ cells in a population.

### SUMMARY OF THE INVENTION

A single cell PCR (scPCR) assay has been developed and qualified to detect individual cells with one or more integrations of LVV sequences, thereby enabling the quantification of the % LVV+ cells in a population. Moreover, the assay is shown to be compatible with a free/thaw cycle after transduction.

Disclosed herein are transduction efficiency assays for lentiviral transduction. The assays comprise transducing a population of cells from a sample with a lentiviral vector comprising a polynucleotide encoding a therapeutic gene; culturing the transduced cells for a period of at least three days; assaying the cultured transduced cells using single cell PCR; measuring presence of genomic and viral DNA sequences in the cells in the sample; quantifying number of transduced cells, wherein cells are considered transduced when they include both genomic and viral DNA sequences; quantifying number of untransduced cells, wherein cells are considered untransduced when they include only genomic DNA sequences; and calculating efficiency of the lentiviral vector transduction (percentage of transduced cells), wherein the efficiency of the transduction is measured as:

$$Transduction efficiency ( \% ) = \frac{\sum transduced cells}{\sum transduced and untransduced cells} \times 100.$$

In some embodiments, the cells are peripheral blood mononuclear cells (PBMCs). The cells may be PBMCs isolated from a subject that has cancer (e.g., multiple myeloma). In some embodiments, the lentiviral vector comprises an engineered antigen receptor. The engineered antigen receptor may be selected from the group consisting of: an engineered αβ-TCR, an engineered δγ-TCR, a chimeric antigen receptor (CAR), and a dimerizing agent regulated immunoreceptor complex (DARIC). In some embodiments, the engineered antigen receptor is an anti-BCMA CAR.

In some embodiments, the cells are hematopoietic stem or progenitor cells. In some embodiments, the assay further comprises obtaining the hematopoietic stem or progenitor cells from a subject that has sickle cell disease or β-thalassemia. In some embodiments, the hematopoietic stem or progenitor cells comprise CD34+ cells, CD133⁺ cells, or CD34⁺CD38^{Lo}CD90⁺CD45RA⁻ cells. In some embodiments, the hematopoietic stem or progenitor cells comprise a pair of β-globin alleles selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, and β⁺/β⁺.

In some embodiments, the polynucleotide encodes a globin selected from the group consisting of a human β-globin, a human δ-globin, an anti-sickling globin, a human γ-globin, a human β^{A-T87Q}-globin, a human β^{A-G16D/E22A/T87Q}-globin, and a human β^{A-T87Q/K95E/K120E}-globin protein. In some embodiments, the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, a G9 vector, a BCL11A shmir vector, or a derivative thereof.

In some embodiments, the culturing of the transduced cells occurs for a period of 3 to 10 days. In some embodiments, a cell is considered as transduced when it is measured as having a Threshold Cycle (Cₜ) value of ≤32 for both genomic and viral DNA sequences. In some embodiments, the viral DNA sequence is a lentiviral vector psi-gag DNA sequence. In some embodiments, the genomic DNA sequence is a RNAseP DNA sequence.

Also disclosed herein are transduction efficiency assays for lentiviral transduction. The assays comprise obtaining a peripheral blood or bone marrow sample from a subject; isolating nucleated cells from the peripheral blood by density gradient centrifugation, e.g., using ficoll; assaying the isolated cells using single cell PCR; measuring presence of genomic and viral DNA sequences in the cells in the sample; quantifying number of transduced cells, wherein cells are considered transduced when they include both genomic and viral DNA sequences; quantifying number of untransduced cells, wherein cells are considered untransduced when they include only genomic DNA sequences; and calculating efficiency of a lentiviral vector transduction, wherein the efficiency of the transduction is measured as:

$$Transduction efficiency ( \% ) = \frac{\sum transduced cells}{\sum transduced and untransduced cells} \times 100.$$

In some embodiments, the nucleated cells are peripheral blood mononuclear cells (PBMCs). In some embodiments, the nucleated cells are hematopoietic stem or progenitor cells. In some embodiments, the hematopoietic stem or progenitor cells comprise CD34+ cells, CD133⁺ cells, or CD34⁺CD38^{Lo}CD90⁺CD45RA⁻ cells. In some embodiments, the hematopoietic stem or progenitor cells comprise a pair of β-globin alleles selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, and β⁺/β⁺.

In some embodiments, the peripheral blood is obtained from a subject treated with a drug product comprising a lentiviral vector comprising a polynucleotide encoding a globin. In some embodiments, the globin is a human β-globin, a human δ-globin, an anti-sickling globin, a human γ-globin, a human β^{A-T87Q}-globin, a human β^{A-G16D/E22A/T87Q}-globin, or a human β^{A-T87Q/K95E/K120E}-globin protein. In some embodiments, the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, a G9 vector, a BCL11A shmir vector, or a derivative thereof.

In some embodiments, the nucleated cells are PBMCs isolated from a subject that has cancer (e.g., multiple myeloma). In some embodiments, the lentiviral vector comprises an engineered antigen receptor. The engineered antigen receptor may be selected from the group consisting of: an engineered αβ-TCR, an engineered δγ-TCR, a chimeric antigen receptor (CAR), and a dimerizing agent regulated immunoreceptor complex (DARIC). In some embodiments, the engineered antigen receptor is a MAGEA4 TCR. In some embodiments, the engineered antigen receptor is an anti-BCMA CAR. In some embodiments, the engineered antigen receptor is an anti-B7H3 CAR. In some embodiments, the engineered antigen receptor is an anti-CD19 CAR.

In some embodiments, a cell is considered as transduced when it is measured as having a Threshold Cycle (Cₜ) value of ≤32 for both genomic and viral DNA sequences. In some embodiments, the viral DNA sequence is a lentiviral vector psi-gag DNA sequence. In some embodiments, the genomic DNA sequence is a RNAseP DNA sequence. In some embodiments, the subject has sickle cell disease or β-thalassemia.

Also disclosed herein are methods for measuring transduction efficiency of a lentiviral vector. The methods comprise assaying a population of cells using single cell PCR, wherein the population of cells are transduced with a lentiviral vector comprising a polynucleotide encoding a therapeutic gene; measuring presence of genomic and viral DNA sequences in the cells; quantifying number of transduced cells, wherein cells are considered transduced when they include both genomic and viral DNA sequences; quantifying number of untransduced cells, wherein cells are considered untransduced when they include only genomic DNA sequences; and calculating efficiency of the lentiviral vector transduction, wherein the efficiency of the transduction is measured as:

$$Transduction efficiency ( \% ) = \frac{\sum transduced cells}{\sum transduced and untransduced cells} \times 100.$$

In some embodiments, the cells are peripheral blood mononuclear cells (PBMCs). In some embodiments, the cells are hematopoietic stem or progenitor cells.

In some embodiments, the methods further comprise obtaining the hematopoietic stem or progenitor cells from a subject that has sickle cell disease or β-thalassemia. In some embodiments, the hematopoietic stem or progenitor cells comprise CD34+ cells, CD133⁺ cells, or CD34⁺CD38^{Lo}CD90⁺CD45RA⁻ cells. In some embodiments, the hematopoietic stem or progenitor cells comprise a pair of β-globin alleles selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, and β⁺/β⁺.

In some embodiments, the cells are isolated from peripheral blood obtained from a subject treated with a drug product comprising a lentiviral vector comprising a polynucleotide encoding a globin. In some embodiments, the globin is a human β-globin, a human δ-globin, an anti-sickling globin, a human γ-globin, a human β^{A-T87Q}-globin, a human β^{A-G16D/E22A/T87Q}-globin, or a human β^{A-T87Q/K95E/K120E}-globin protein. In some embodiments, the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, a G9 vector, a BCL11A shmir vector, or a derivative thereof.

In some embodiments, the cells are PBMCs isolated from a subject that has cancer (e.g., multiple myeloma). In some embodiments, the lentiviral vector comprises an engineered antigen receptor. The engineered antigen receptor may be selected from the group consisting of: an engineered αβ-TCR, an engineered δγ-TCR, a chimeric antigen receptor (CAR), and a dimerizing agent regulated immunoreceptor complex (DARIC). In some embodiments, the engineered antigen receptor is an anti-BCMA CAR.

In some embodiments, a cell is considered as transduced when it is measured as having a Threshold Cycle (Cₜ) value of ≤32 for both genomic and viral DNA sequences. In some embodiments, the viral DNA sequences is a lentiviral vector psi-gag DNA sequence. In some embodiments, the genomic DNA sequence is a RNAse P DNA sequence.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 provides a schematic detailing a single cell transduction efficiency assay. Transduced cells are cultured in vitro for 3 or more days to remove non-integrated LVV. Cells are then arrayed into a 96-well plate using a flow cytometer and lysed with proteinase K. Pre-amplification mix is then added using the VIAFLO electronic pipette, and PsiGag and RNAseP regions are amplified by PCR. The preamplified product can be stored at −20° C. Up to four pre-amplified 96-well plates can be combined onto a single 384-well plate via the VIAFLO, and assayed by TaqMan qPCR for presence or absence of RNAseP and PsiGag sequences to calculate transduction efficiency. System suitability controls, along with sample and assay acceptance controls, are indicated where appropriate.

FIG. 2 shows an FACS sorting strategy for single viable cells. Cell populations are separated from debris using a FSC-Area/SSC-Area gate, singlets gated by FSC-Area/FSC-Height, and viable cells gated on APC-Cy7.

FIGS. 3A-3B show semi-nested PCR/qPCR for PsiGag (FIG. 3A) and RNAseP (FIG. 3B). NF: nested forward primer. NR: nested reverse primer. F: forward primer. R: reverse primer.

FIG. 4 shows distribution of single cell Ct measurements from 11 plates (1012 single cell data points) of positive and negative controls (Table 1). The signal presence/absence cut-off is set at Ct=32. Measurements where no amplification occurred (undetermined Ct) are assigned a Ct of 41. Only 7.4% of the analyzed data points had RNAseP Ct>32.

FIG. 5 shows margin of error in using a subset of sampled cells to estimate the actual transduction efficiency in a sample. The margin of error depends on the measured proportion of marked cells, with maximum margin of error occurring at 50% TE. The margin of error in measuring three hypothetical levels of TE with 95% confidence is shown.

FIG. 6 shows change in transduction efficiency during in vitro culture. TE was measured by single cell PCR from triplicate transductions either under standard conditions or alternative transduction methods. p-value: *0.03; ns=not significant.

FIGS. 7A-7D demonstrate confirmation of assay accuracy by comparing single cell PCR results to FACS on cells transduced with LentiGFP. CD34+ cells were transduced with LentiGFP and cultured in SCGM growth media for 4-7 days. The percentage of GFP+ viable singlets was determined by FACS (FIG. 7A). FIG. 7B shows linear correlation between percent GFP+ observed by FACS and percent PsiGag+ measured by single cell PCR. Slope: 1.08±0.07 Y-intercept: −4.78±3.37 (FIGS. 7C-7D). Index FACS sorting records FACS data for each cell deposited into the 96 well plate for single cell PCR. The GFP expression of cells identified as transduced is shown as circles in FIG. 7C, and location of GFP+ cells in a 96-well plate is shown in FIG. 7D. Arrows indicate two cells that were identified as transduced by single cell PCR but that are not GFP+ by FACS.

FIG. 8 provides comparison of transduction efficiency readouts from single cell PCR and single colony PCR. Aggregated data is plotted from 17 paired readouts using three lots of CD34+ cells and two lots of LentiGlobin BB 305. Slope: 0.919±0.075. Y-intercept: 14.62±4.455. Dotted lines indicate the 95% CI of the linear fit.

FIGS. 9A-9B demonstrate linearity of measurement by spiking transduced CK3 cells into untransduced CD34+ cells. FIG. 9A shows the composition of CK3 in the sorted sample was verified by FACS, as CK3 cells are GFP+. FIG. 9B provides linear regression of the data. Slope: 0.949±0.021. Y-intercept: −0.723±1.154. Dotted lines indicate the 95% CI of the linear fit.

FIGS. 10A-10D demonstrate assay and sample acceptance criteria. FIG. 10A shows Closed circles: PsiGag Ct from 10-cell wells is negatively correlated with transduction efficiency measured for the sample, with a slope of −0.053. Open circles: 10-cell PsiGag Ct after linear adjustment for TE in the sample. FIG. 10B shows RNAseP Ct from 10-cell wells is not correlated with TE and does not require adjustment. FIG. 10C provides measured Cts for all six assay controls. 10-cell PsiGag Ct is adjusted as in FIG. 10A. Dashed lines indicate the upper and lower acceptance limit at three standard deviations from the mean. FIG. 10D provides percent of samples negative for both PsiGag and RNAseP (no amplification) or positive for PsiGag but negative for genomic RNAseP (PsiGag only). Dashed lines indicate the upper acceptance limit at three standard deviations from the mean.

FIGS. 11A-11B demonstrate measurement of transduction efficiency in HSCs. CD34+ cells were transduced with LentiGlobin. FIG. 11A shows that following transduction, cells were washed and FACS-stained for CD34+, CD38, CD90, and CD45RA. Cells were FACS-sorted either from the singlet gate (bulk sample) or from the CD34⁺CD38^{lo}CD90⁺CD45RA⁻ gate (HSC gate). FIG. 11B shows that after 4 days in culture, transduction efficiency was determined by single cell PCR assay.

FIGS. 12A-12D demonstrate differences between vector copy number and transduction efficiency. FIGS. 12A-12B provide a comparison of transduction efficiency measured by single cell PCR to bulk VCN at day 7 (FIG. 12A) and day 14 (FIG. 12B). Data points are color-coded based on different transduction methods. The Poisson distribution is calculated using Equation 4. Dotted gridlines indicated VCN of 0.3 and 1.0. FIGS. 12C-12D provide a comparison of transduction efficiency estimated from day 7 VCN using Equation 5 to the transduction efficiency measured by single cell PCR. Mean difference: −23.19%, paired t-test p<0.0001 (FIG. 12C). Mean difference: −16.61%, paired t-test p<0.0001 (FIG. 12D).

FIG. 13 provides identification of outliers by paired measurements of transduction efficiency and bulk vector copy number at day 7. The Poisson distribution is calculated using Equation 4 and plotted as a solid line. The best fit of the Poisson distribution to the dataset (n=40) resulted in k=0.48, plotted as a dashed line. The 95% prediction interval of the fit is plotted as a dotted line. Arrow: data point outside the prediction interval.

FIG. 14 provides a schematic detailing a peripheral blood single cell PCR assay. Peripheral blood samples were subjected to density gradient centrifugation using ficoll to collect nucleated cells. Cells were stained with antibodies, and desired populations were single cell sorted into 96-well plates using the Sony SH800Z flow cytometer. Cells were lysed with proteinase K and PsiGag and RNAseP sequences were amplified using AmpliTaq Gold PCR. Up to four pre-amplified 96-well plates were combined onto a single 384-well plate using the VIAFLO, and assayed by TaqMan qPCR for presence or absence or RNAseP and PsiGag sequences to calculate % LVV+ cells.

FIG. 15 demonstrates FACS sorting strategy for single DNA+ cells. Cell populations are separated from debris using a FSC-Area/SSC-Area gate, singlets gated by FSC-Area/FSC-Height, and DNA+ cells gated on PerCyp5.5-Area (Draq5).

FIG. 16 demonstrates FACS sorting strategy for CD45+CD34+, CD45+CD3− and CD45+CD3+ cells. Debris was eliminated in the “All Events” FSC-A/BSC-A plot and doublets were excluded in the FSC-A/FSC-H “Cells” plot. From the singlet population, CD45+ live cells were gated on Brilliant Violet-A/PE-Cy7-A. From the CD45+ live population, DNA+ cells were gated on PerCP-Cy5.5-A/PE-Cy7-A. From the DNA+ population CD3−, CD3+, and CD34+ PBMCs were gated on PE-A/APC-A.

FIG. 17 demonstrates a gating strategy for demonstrating linearity. One 96-well plate was sorted from each gate for % LVV+ determination by scPCR.

FIG. 18 provides a linear correlation between % GFP+ observed by FACS and % LVV+ measured by scPCR. Slope: 0.98±0.011, Y-intercept: −0.29±0.70. Dotted lines indicate the 95% CI of the linear fit.

### DETAILED DESCRIPTION OF THE INVENTION

An assay that can quantify the transduction efficiency of a lentiviral vector in a population of cells is described herein. Moreover, this assay can be used to measure the percent of cells transduced with a lentiviral vector (LVV) in post-infusion samples of peripheral blood. Also disclosed herein are methods for measuring transduction efficiency of a lentiviral vector.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements. The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. As used herein, the terms “include” and “comprise” are used synonymously. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The term “vector” is used herein to refer to a nucleic acid molecule capable of transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., retroviral vectors and lentiviral vectors.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term “viral vector” may refer either to a virus or viral particle capable of transferring a nucleic acid into a cell or to the transferred nucleic acid itself. Viral vectors and transfer plasmids contain structural and/or functional genetic elements that are primarily derived from a virus. The term “lentiviral vector” refers to a retroviral vector or plasmid containing structural and functional genetic elements, or portions thereof, including LTRs that are primarily derived from a lentivirus. The terms “lentiviral vector” and “lentiviral expression vector” may be used to refer to lentiviral transfer plasmids and/or infectious lentiviral particles in particular embodiments. Where reference is made herein to elements such as cloning sites, promoters, regulatory elements, heterologous nucleic acids, etc., it is to be understood that the sequences of these elements are present in RNA form in the lentiviral particles contemplated herein and are present in DNA form in the DNA plasmids contemplated herein.

“Transfection” refers to the process of introducing naked DNA into cells by non-viral methods.

“Infection” refers to the process of introducing foreign DNA into cells using a viral vector. “Transduction” refers to the introduction of foreign DNA into a cell's genome using a viral vector.

“Vector copy number” or “VCN” refers to the number of copies of a vector, or portion thereof, in a cell's genome. The average VCN may be determined from a population of cells or from individual cell colonies.

“Transduction efficiency” refers to the percentage of cells transduced with at least one copy of a vector. For example if 1 x 10⁶ cells are exposed to a virus and 0.5×10⁶ cells are determined to have a least one copy of a virus in their genome, then the transduction efficiency is 50%. Transduction efficiency and % lentiviral vector positive (% LLV+) are used interchangeably.

The term “globin” as used herein refers to proteins or protein subunits that are capable of covalently or noncovalently binding a heme moiety, and can therefore transport or store oxygen. Subunits of vertebrate and invertebrate hemoglobins, vertebrate and invertebrate myoglobins or mutants thereof are included by the term globin. The term excludes hemocyanins. Examples of globins include α-globin or variants thereof, β-globin or variants thereof, a γ-globin or variants thereof, and δ-globin or variants thereof.

Additional definitions are set forth throughout this disclosure.

In the following description, certain specific details are set forth in order to provide a thorough understanding of various illustrative embodiments of the invention contemplated herein. However, one skilled in the art will understand that particular illustrative embodiments may be practiced without these details.

### Transduction Efficiency Assays

Disclosed herein are transduction efficiency assays for viral transduction (e.g., lentiviral transduction). In some embodiments a transduction efficiency assay comprises transducing a population of cells from a sample with a lentiviral vector comprising a polynucleotide encoding a therapeutic gene; culturing the transduced cells for a period of at least three days; assaying the cultured transduced cells using single cell PCR; measuring presence of genomic and viral DNA sequences in the cells in the sample; quantifying number of transduced cells, wherein cells are considered transduced when they include both genomic and viral DNA sequences; quantifying number of untransduced cells, wherein cells are considered untransduced when they include only genomic DNA sequences; and calculating efficiency of the lentiviral vector transduction (percentage of transduced cells).

In some embodiments, a transduction efficiency assay for viral transduction (e.g., lentiviral transduction) comprises obtaining a peripheral blood or bone marrow sample from a subject; isolating nucleated cells from the peripheral blood by density gradient centrifugation, e.g., ficoll; assaying the isolated cells using single cell PCR; measuring presence of genomic and viral DNA sequences in the cells in the sample; quantifying number of transduced cells, wherein cells are considered transduced when they include both genomic and viral DNA sequences; quantifying number of untransduced cells, wherein cells are considered untransduced when they include only genomic DNA sequences; and calculating efficiency of a lentiviral vector transduction.

In particular aspects, the efficiency of the transduction is measured as

$$Transduction efficiency ( \% ) = \frac{\sum transduced cells}{\sum transduced and untransduced cells} \times 100.$$

In some aspects, a cell is considered transduced when it is measured as having a Threshold Cycle (Cₜ) value of ≤32 for both genomic and viral DNA sequences. A viral DNA sequence may be a lentiviral vector psi-gag DNA sequence (e.g., present in LentiD, LentiG, bb2121, LentiGFP, etc.). A genomic DNA sequence may be a RNAseP DNA sequence. In some aspects, a cell is considered untransduced when it is measured as having a Threshold Cycle (Ct) value of >32 for a viral DNA sequence and a Ct value of ≤32 for a genomic DNA sequence.

In some aspects, cells (e.g., from a population of cells or nucleated cells) are peripheral blood mononuclear cells or hematopoietic cells. In some aspects, cells (e.g., from a population of cells or nucleated cells) are peripheral blood mononuclear cells (PBMCs). In particular aspects, PBMCs are isolated from a subject that has cancer. In some embodiments, PBMCs are isolated from a subject that has multiple myeloma, leukemia, or lymphoma.

In some aspects, a lentiviral vector includes an engineered antigen receptor. The engineered antigen receptor may be an engineered α⊕-TCR, an engineered δγ-TCR, a chimeric antigen receptor (CAR), or a dimerizing agent regulated immunoreceptor complex (DARIC). In certain aspects, the engineered antigen receptor is an engineered αβ-TCR, e.g., a MAGEA4 TCR. In certain aspects, the engineered antigen receptor is an engineered δγ-TCR. In certain aspects, the engineered antigen receptor is a CAR. In certain aspects, the engineered antigen receptor is a DARIC. In certain aspects, the engineered antigen receptor is an anti-BCMA, anti-B7H3, or anti-CD19 CAR.

In some aspects, cells (e.g., from a population of cells or nucleated cells) are hematopoietic stem or progenitor cells. In some aspects, the method comprises obtaining a sample of hematopoietic stem or progenitor cells from a subject that has sickle cell disease or β-thalassemia. Suitable methods for obtaining hematopoietic stem or progenitor cells from a subject include apheresis.

In some aspects hematopoietic stem or progenitor cells are selected from the group consisting of CD34⁺ cells, CD133⁺ cells, CD34⁺CD133⁺ cells, CD34⁺CD38^{Lo}CD90⁺CD45RA⁻ cells, and combinations thereof. In certain aspects, the hematopoietic stem or progenitor cells include CD34⁺ cells. In certain aspects, the hematopoietic stem or progenitor cells include CD133⁺ cells. In certain aspects, the hematopoietic stem or progenitor cells include CD34⁺CD133⁺ cells. In certain aspects, the hematopoietic stem or progenitor cells include CD34⁺CD38^{Lo}CD90⁺CD45RA⁻ cells.

In some aspects, the hematopoietic stem or progenitor cells comprise a pair of β-globin alleles selected from the group consisting of β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, and β⁺/β⁺. In certain aspects, the hematopoietic stem or progenitor cells comprise a pair of β-globin alleles that are β^{E}/β⁰. In certain aspects, the hematopoietic stem or progenitor cells comprise a pair of β-globin alleles that are β^{C}/β⁰. In certain aspects, the hematopoietic stem or progenitor cells comprise a pair of β-globin alleles that are β⁰/β⁰. In certain aspects, the hematopoietic stem or progenitor cells comprise a pair of β-globin alleles that are β^{C}/β^{C}. In certain aspects, the hematopoietic stem or progenitor cells comprise a pair of β-globin alleles that are β^{E}/β^{E}. In certain aspects, the hematopoietic stem or progenitor cells comprise a pair of β-globin alleles that are β^{E}/β⁺. In certain aspects, the hematopoietic stem or progenitor cells comprise a pair of β-globin alleles that are β^{C}/β^{E}. In certain aspects, the hematopoietic stem or progenitor cells comprise a pair of β-globin alleles that are β^{C}/β⁺. In certain aspects, the hematopoietic stem or progenitor cells comprise a pair of β-globin alleles that are β⁰/β⁺. In certain aspects, the hematopoietic stem or progenitor cells comprise a pair of β-globin alleles that are β^{E}/β^{E}. In certain aspects, the hematopoietic stem or progenitor cells comprise a pair of β-globin alleles that are β⁺/β⁺.

In some embodiments, a population of cells is transduced with a vector (e.g., a lentiviral vector) comprising a polynucleotide encoding a therapeutic gene. In some embodiments, a population of cells is transduced with a vector (e.g., a lentiviral vector) comprising a polynucleotide encoding a globin. In some embodiments, a subject is treated with a drug product comprising a lentiviral vector comprising a polynucleotide encoding a globin. In some aspects, the globin is a human β-globin, a human δ-globin, an anti-sickling globin, a human γ-globin, a human β^{A-T87Q}-globin, a human β^{A-G16D/E22A/T87Q}-globin, or a human β^{A-T87Q/K95E/K120E}-globin protein. In certain aspects, the globin is a human β-globin protein. In certain aspects, the globin is a human δ-globin protein. In certain aspects, the globin is an anti-sickling globin protein. In certain aspects, the globin is a human γ-globin protein. In certain aspects, the globin is a human β^{A-T87Q}-globin protein. In certain aspects, the globin is a human β^{A-G16D/E22A/T87Q}-globin protein. In certain aspects, the globin is a human β^{A-T87Q/K95E/K120E}-globin protein.

In some embodiments, the vector is a lentiviral vector. In some aspects the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, a G9 vector, a BCL11A shmir vector, or a derivative thereof. In some aspects, the lentiviral vector is an AnkT9W vector or a derivative thereof. In some aspects, the lentiviral vector is a T9Ank2W vector or a derivative thereof. In some aspects, the lentiviral vector is a TNS9 vector or a derivative thereof. In some aspects, the lentiviral vector is a TNS9.3 vector or a derivative thereof. In some aspects, the lentiviral vector is a TNS9.3.55 vector or a derivative thereof. In some aspects, the lentiviral vector is a lentiglobin HPV569 vector or a derivative thereof. In some aspects, the lentiviral vector is a lentiglobin BB305 vector or a derivative thereof. In some aspects, the lentiviral vector is a BG-1 vector or a derivative thereof. In some aspects, the lentiviral vector is a BGM-1 vector or a derivative thereof. In some aspects, the lentiviral vector is a d432βAγ vector or a derivative thereof. In some aspects, the lentiviral vector is a mLARβΔγV5 vector or a derivative thereof. In some aspects, the lentiviral vector is a GLOBE vector or a derivative thereof. In some aspects, the lentiviral vector is a G-GLOBE vector or a derivative thereof. In some aspects, the lentiviral vector is a βAS3-FB vector or a derivative thereof. In some aspects, the lentiviral vector is a V5 vector or a derivative thereof. In some aspects, the lentiviral vector is a V5m3 vector or a derivative thereof. In some aspects, the lentiviral vector is a V5m3-400 vector or a derivative thereof. In some aspects, the lentiviral vector is a G9 vector or a derivative thereof. In some aspects, the lentiviral vector is a BCL11A shmir vector or a derivative thereof.

In some aspects, the transduced cells are cultured for a period of at least three days. In some aspects, the culturing of the transduced cells occurs for a period of 3 to 14 days. In some aspects, the culturing of the transduced cells occurs for a period of 3 to 10 days. In some aspects, the culturing of the transduced cells occurs for a period of 3 to 7 days. In some aspects, the culturing of the transduced cells occurs for a period of 3 to 6 days. In some aspects, the culturing of the transduced cells occurs for a period of 4 to 7 days. The cells may be cultured in growth media (e.g., stem cell growth media).

### Methods for Measuring Potency of a Drug Product

Also disclosed herein are methods for measuring transduction efficiency of a viral vector (e.g., a lentiviral vector). In some aspects, the methods comprise assaying a population of cells using single cell PCR, wherein the population of cells are transduced with a lentiviral vector comprising a polynucleotide encoding a therapeutic gene; measuring presence of genomic and viral DNA sequences in the cells; quantifying number of transduced cells, wherein cells are considered transduced when they include both genomic and viral DNA sequences; quantifying number of untransduced cells, wherein cells are considered untransduced when they include only genomic DNA sequences; and calculating efficiency of the lentiviral vector transduction.

In particular aspects, the efficiency of the transduction is calculated as

$$Transduction efficiency ( \% ) = \frac{\sum transduced cells}{\sum transduced and untransduced cells} \times 100.$$

In some aspects, a cell is considered transduced when it is measured as having a Threshold Cycle (CO value of ≤32 for both genomic and viral DNA sequences. A viral DNA sequence may be a lentiviral vector psi-gag DNA sequence (e.g., found in LentiD, LentiG, bb2121, LentiGFP, etc.). A genomic DNA sequence may be a RNAseP DNA sequence. In some aspects, a cell is considered untransduced when it is measured as having a Threshold Cycle (Ct) value of >32 for a viral DNA sequence and a Ct value of ≤32 for a genomic DNA sequence.

In some aspects, the cells are peripheral blood mononuclear cells or hematopoietic cells. In some aspects, the cells are hematopoietic stem or progenitor cells. In some aspects, the method comprises obtaining a sample of hematopoietic stem or progenitor cells from a subject that has sickle cell disease or β-thalassemia. In some aspects hematopoietic stem or progenitor cells are selected from the group consisting of CD34⁺ cells, CD133⁺ cells, CD34⁺CD133⁺ cells, or CD34⁺CD38^{Lo}CD90⁺CD45RA⁻ cells. In some aspects, the hematopoietic stem or progenitor cells comprise a pair of β-globin alleles selected from the group consisting of β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, and β⁺/β⁺.

In some aspects, the cells are isolated from peripheral blood obtained from a subject treated with a drug product comprising a lentiviral vector comprising a polynucleotide encoding a globin. The globin may be a human β-globin, a human δ-globin, an anti-sickling globin, a human γ-globin, a human β^{A-T87Q}-globin, a human β^{A-G16D/E22A/T87Q}-globin, or a human β^{A-T87Q/K95E/K120E}-globin protein. The lentiviral vector may be an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, a G9 vector, a BCL11A shmir vector, or a derivative thereof.

In some aspects, the cells are peripheral blood mononuclear cells (PBMCs). In particular aspects, PBMCs are isolated from a subject that has cancer (e.g., multiple myeloma). In some aspects, a lentiviral vector includes an engineered antigen receptor. The engineered antigen receptor may be an engineered αβ-TCR, an engineered δγ-TCR, a chimeric antigen receptor (CAR), or a dimerizing agent regulated immunoreceptor complex (DARIC). In particular aspects, the engineered antigen receptor is an anti-BCMA CAR.

All publications, patent applications, and issued patents cited in this specification are herein incorporated by reference as if each individual publication, patent application, or issued patent were specifically and individually indicated to be incorporated by reference.

The following examples are provided by way of illustration only and not by way of limitation. Those of skill in the art will readily recognize a variety of noncritical parameters that could be changed or modified in particular embodiments to yield essentially similar results.

### EXEMPLIFICATION

### Example 1

### Development of Single Cell PCR Assay to Quantify the Transduction Efficiency of a Lentiviral Vector

Transduction efficiency (TE) is defined as a percentage of cells with at least one integration of a lentiviral vector (LVV) (e.g., LentiGlobin BB305 LVV). High TE is crucial to the efficacy of autologous gene therapy, as transplantation of cells with a low TE, regardless of their per cell vector copy number (VCN), results in a graft with fewer gene-modified stem and progenitor cells that can beneficially contribute to treating transfusion-dependent β-Thalassemia. TE is typically measured by FACS; however the erythroid-specific promoter in LentiGlobin precludes expression of HBB-T87Q in the CD34+ drug product. Instead, drug product cells are cultured to remove non-integrated LVV DNA sequences, and from their pooled genomic prep, the quantity of PsiGag sequences are determined by qPCR and divided by the quantity of a genomic reference sequence, RNAseP, to calculate VCN. It is assumed that the per-cell distribution of LVV integrations follows a Poisson curve, and TE can be estimated from pooled cell VCN. For example, at VCN=1, 63% of cells are expected to have at least one LVV integration. In practice, the TE is variable and often lower than expected, and an assay is needed to directly measure the percentage of transduced cells in the LentiGlobin drug products.

An alternative method to measure transduction efficiency is to plate drug product cells in Methocult, and after 1-2 weeks, pick colonies that develop from single colony-forming cells to measure their VCN by TaqMan® qPCR. The TE can then be calculated as a percent of colonies with VCN≥1. Although TaqMan qPCR lacks sensitivity to assay single cell DNA inputs, most hematopoietic colonies provide a sufficient template. The single colony qPCR assay has two main drawbacks. First, manual picking of single colonies from Methocult plates is imprecise, labor-intensive, and difficult to control. Second, on average only 25% of drug product CD34+ cells are colony forming, while important cells like the hematopoietic stem cells (HSCs) do not form colonies in Methocult and therefore cannot be assayed.

Single cell PCR is an alternative to FACS or single colony qPCR for quantifying transduction efficiency without the need for transgene expression. Single cells can be reliably arrayed and lysed in a 96-well plate using a modern FACS sorter. When amplifying two targets, a viral-specific target and a genomic reference target, the semi-nested PCR approach reduces primer pool complexity from 8 primers to 6, reducing the likelihood of cross-primer interactions. This report describes a novel assay that utilizes semi-nested PCR/qPCR to measure the percentage of cells in a sample that are transduced with at least one copy of LentiGlobin transgene. Due to non-linear PCR amplification, the assay only measures TE and not per cell VCN.

### Results

### Sensitivity and Specificity in Control Cell Lines, Establishment of Analysis Thresholds, False Positive and Negative Rates

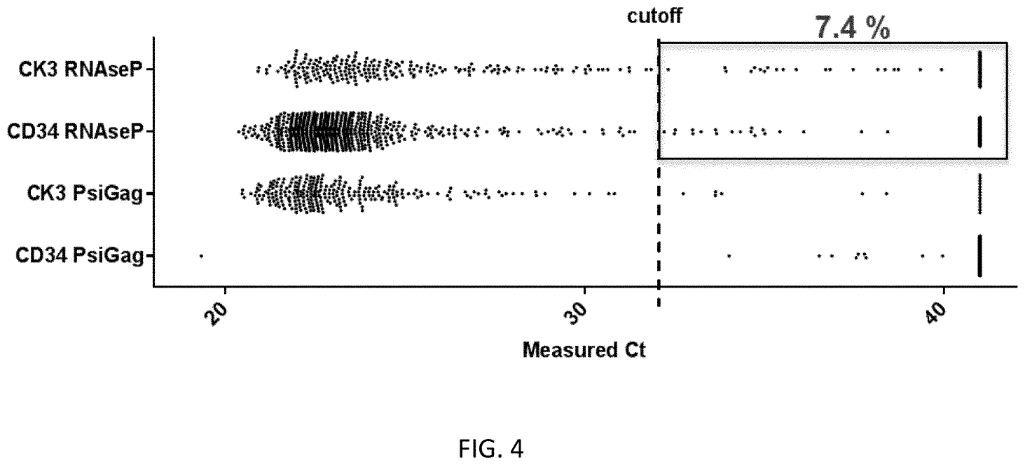

In this assay, integrated viral PsiGag DNA sequence and genomic RNAseP DNA sequence were preamplified by PCR from individual cell lysates arrayed in a 96-well plate. The preamplified material was then analyzed by 40 cycles of qPCR for presence or absence of PsiGag and RNAseP. The qPCR Cts from replicate plates of CK3 cells (theoretically 100% transduced) and untransduced CD34+ cells are shown in FIG. 4. Wells with undetermined Ct, indicating no qPCR amplification signal, were assigned a Ct of 41. Of wells with Ct<41, the average PsiGag Ct was 23.43, whereas the average RNAseP Ct was 24.03 (Table 1). A cut-off of Ct=32.00 was chosen for both PsiGag and RNAseP Ct measurements, based on the PsiGag mean Ct plus 3 standard deviations (Table 1), and consistent with Ct cut-off used in single colony qPCR assay. This cut-off is used to make one of four possible determinations for each sampled single cell well (Table 2). Of the 1012 single cell data points on the 11 analyzed plates, only 74 had RNAseP Ct>32 (FIG. 4), indicating a 92.7% efficiency in arraying single cells and successfully amplifying their genomes. The transduction efficiency is calculated using Equation 2:

$$Transduction efficiency ( \% ) = \frac{\sum transduced cells}{\sum transduced and untransduced cells} \times 100 \qquad ( Equation 2 )$$

Of the 216 CK3 sample wells, 94.98% were marked, indicating a 5.02% false negative rate as the CK3 clonal cell line is 100% transduced (Table 3). Of the 899 untransduced CD34+ sample wells, only 0.17% were marked, indicating a 0.17% false positive rate.

TABLE 1
Distribution of single cell Ct measurements
from successful qPCR reactions. Ct values
where no amplification occurred (undetermined
Ct) were excluded from analysis.

| | Data | Mean | | % | Mean Ct + |
|---|---|---|---|---|---|
| qPCR region | points | Ct | StDev | CV | 3 StDev |
| PsiGag (transgene DNA) | 265 | 23.43 | 2.60 | 11.12 | 31.24 |
| RNAseP (genomic DNA) | 899 | 24.03 | 3.15 | 13.11 | 33.48 |

TABLE 2
Four possible data interpretations made from the multiplexed
qPCR measurement in each well.

| qPCR result | Interpretation |
|---|---|
| RNAseP Ct ≤ 32, PsiGag Ct ≤ 32 | Transduced cell |
| RNAseP Ct ≤ 32, PsiGag Ct > 32 or | Untransduced cell |
| undetermined | |
| RNAseP Ct > 32 or undetermined, | Empty well, ignored from |
| PsiGag Ct > 32 or undetermined | analysis |
| RNAseP Ct > 32 or undetermined, | RNAseP amplification failure, |
| PsiGag Ct ≤ 32 | ignored from analysis |

TABLE 3
Assay accuracy and false discovery rates. Single cell data
set was generated from three plates of CK3 cells and 8 plates of
un-transduced CD34+ cells. Calculated false negative rate: 5.02%.
Calculated false positive rate: 0.17%.

| | | | | Measured | | |
|---|---|---|---|---|---|---|
| | Control | # | Data | TE | | |
| Sample | type | plates | points | (%) | StDev | FDR |
| CK3 cell line | positive | 3 | 216 | 94.98 | 0.898 | 5.02 |
| CD34+ mPB | negative | 8 | 644 | 0.17 | 0.478 | 0.17 |
| (untransduced) | | | | | | |

### Margin of Error of the Assay vs Number of Cells Sampled

With the assay run in 96-well format, accounting for control wells and wells excluded from analysis (Table 2), the transduction efficiency was calculated, on average, from 80 single cell data points. The margin of error formula was used to estimate the statistical error in determining the TE in the assayed sample from measurements made in a subset of single cells (Equation 3).

$$Margin of error = z * \sqrt{\frac{p ( 1 - p )}{n}} \qquad ( Equation 3 )$$

(p=proportion of transduced cells, n=observed sample size, z=1.96 for 95% C.I.)

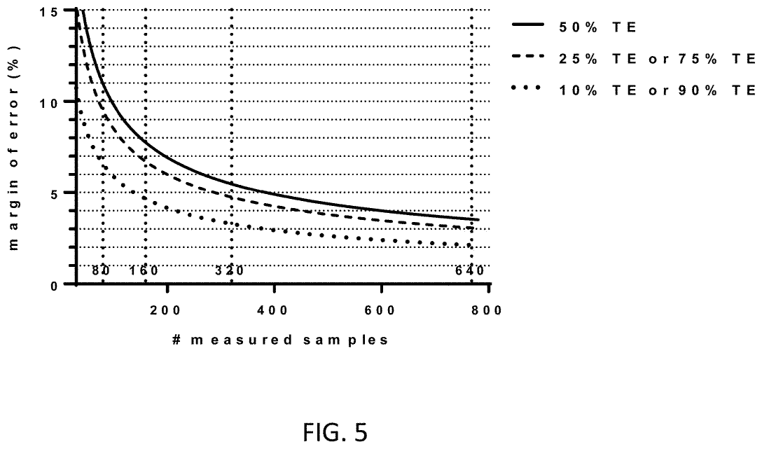

Plotting the margin of error against observed sample size at three hypothetical levels of TE gives an estimate of statistical error from analyzing 1, 4, or 8 96-well plates per sample (FIG. 5). Measuring one 96-plate of cells has an estimated margin of error of ±7-11%, depending on measured TE. Increasing the assay size to two 96-well plates per sample reduces the margin of error to ±4.5-8%. Increasing the assay size to four 96-well plates per sample reduces the margin of error to ±3-6.5%. Unless otherwise specified, the TE values in this report are based on a single 96-well plate per sample.

### Stabilization in TE During Post-Transduction in Vitro Culture

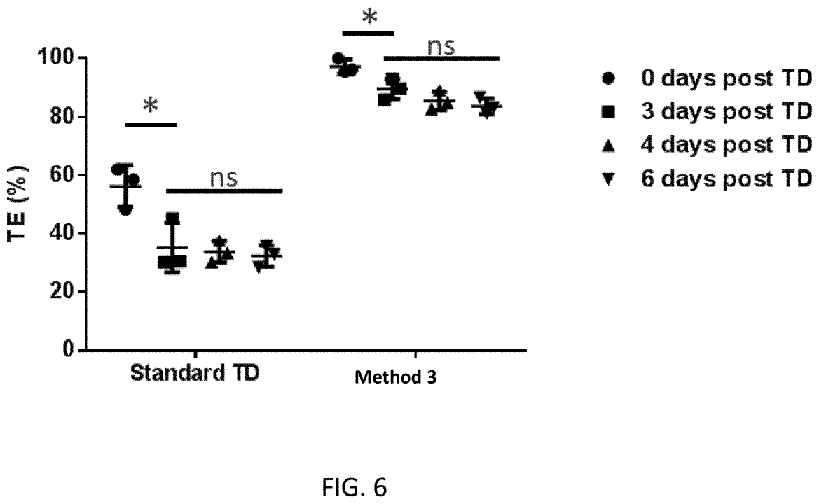

To investigate the earliest readout for this assay, CD34+ cells were transduced in triplicate with LentiGlobin BB305 lentiviral vector (MOI 48.3) utilizing either the standard transduction method or alternative transduction methods. A high MOI was chosen to load the cells with the maximum amount of LVV. Following transduction, cells were washed and cultured in SCGM growth media, and transduction efficiency was measured by single cell PCR after 0, 3, 4, and 6 days (FIG. 6). For all transduction methods, the TE was higher immediately post transduction, likely due to the presence of non-integrated LVV DNA. No statistically significant difference was observed in TE measurements between 3 and 6 days in culture.

### Minimum Cell Number Required to Fill an Assay Plate

To measure the minimum number of transduced cells required for this assay, 5,000 transduced CD34+ cells transduced with LentiGlobin BB305 lentiviral vector were plated in SCGM growth media in a 96-well flat bottom plate (5e4 viable cells/ml, 100 μL). After 4 days in culture, cells were transferred to 1.2 ml microtiter FACS tubes and arrayed into assay plates. The volume of cells left over after FACS-sorting was then measured to determine the fraction of the sample used for FACS. In both cases, 5,000 transduced cells were sufficient to fill a 96-well assay plate, including triplicate 10-cell control wells (Table 4).

TABLE 4
Minimum number of transduced cells required to fill a single
cell PCR assay plate.

| | Starting quantity of | Fraction of sample used to |
|---|---|---|
| Replicate | transduced cells | fill an assay plate |
| 1 | 5e3 | 0.4 |
| 2 | 5e3 | 0.4 |
| 3 | 4e4 | 0.3 |

### Effect of Sample Cryopreservation on Assay Readout

CD34+ cells transduced with LentiGlobin BB305 lentiviral vector were cultured for 6 days in SCGM growth media and analyzed by single cell PCR (Table 5). Left over cells from each sample were then cryopreserved in CryoStor 10 (CS10), then thawed in SCGM containing DNAse, and re-analyzed by single cell PCR. Freeze/thawing the cells had a negligible effect on observed TE, well within the margin of error of the assay.

TABLE 5
Effect of sample cryopreservation on assay readout.

| Sample | TE (%, fresh) | TE (%, after freeze/thaw) | difference |
|---|---|---|---|
| 1 | 96.20 | 96.39 | 0.19% |
| 2 | 93.02 | 94.05 | 1.03% |
| 3 | 86.42 | 87.06 | 0.64% |
| 4 | 87.21 | 89.77 | 2.56% |

### Assay Accuracy by Comparison of TE Measurements by Single Cell PCR and FACS

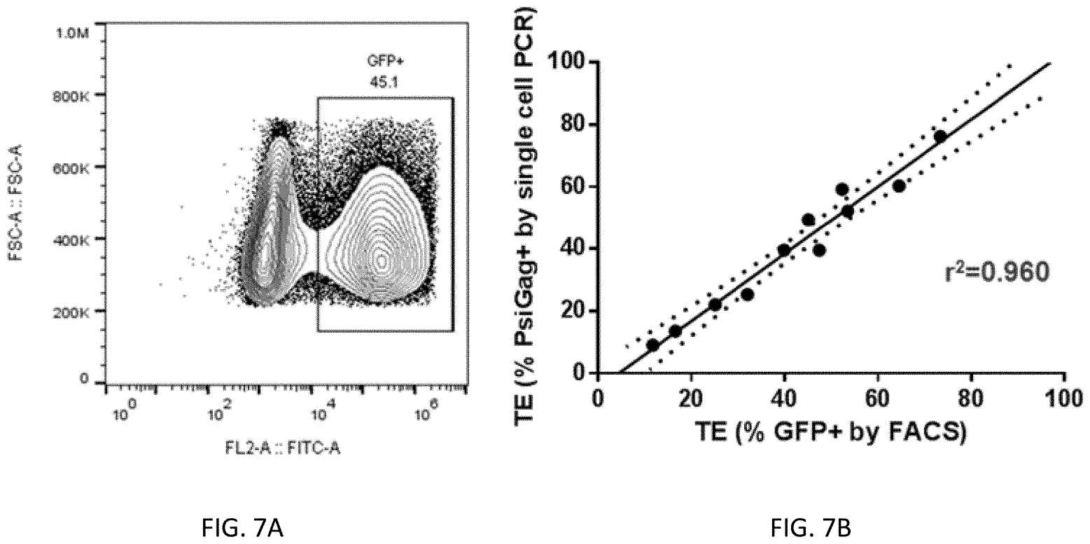

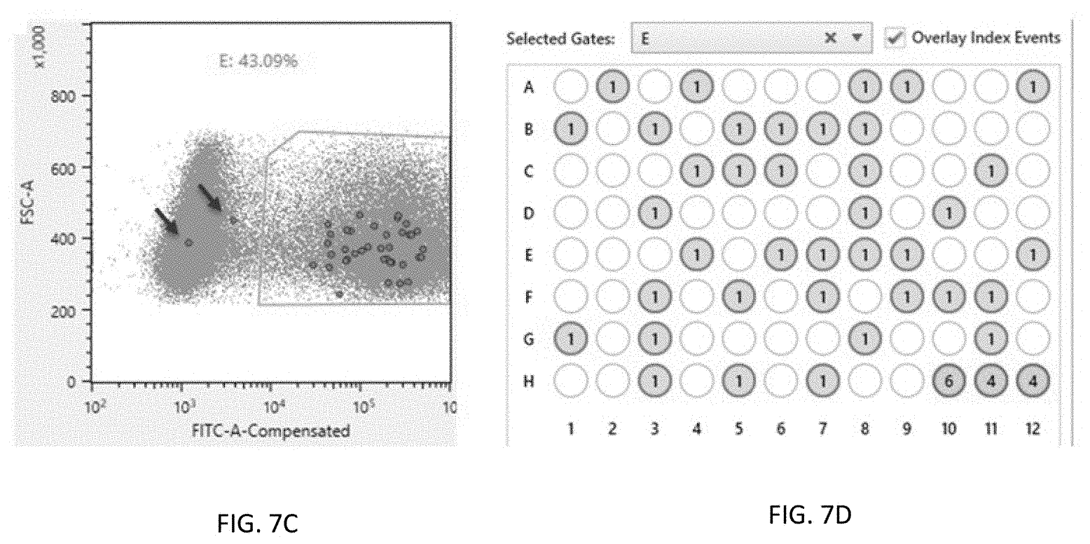

Assay accuracy was evaluated using a GFP LVV by comparing the transduction efficiency observed by single cell PCR for PsiGag to percent GFP+ by FACS. CD34+ cells were transduced with GFP LVV and cultured in SCGM growth media for 4-7 days. TE was determined by single cell PCR and compared to the percent GFP+ measured by FACS (FIGS. 7A-7B, linear fit r²=0.960). The readout was linear (slope=1.08) and Y-intercept of −4.78 is consistent with the measured 5% false negative rate.

Accuracy was further confirmed by index sorting. In an index sort, the fluorescence profile of each arrayed cell is recorded, allowing traceability of each sorted cell in the assay plate (FIGS. 6C-6D). In principle, all GFP+ cells should also be positive for PsiGag by single cell PCR, whereas all GFP− cells should be untransduced. For example, the cell arrayed into well A1 is GFP− (FIGS. 7C-7D), and the qPCR data for well Al should be RNAseP+ and PsiGag−, indicating an untransduced cell (Table 2). By comparison of index sorting data to qPCR data, 95.57% of GFP+ cells were identified as transduced (Table 6), consistent with the 5% false negative rate measured in CK3 cell line (Table 3). 5.09% of GFP− cells were identified as transduced with LentiGFP, a much higher percentage than the false positive rate observed in untransduced cells (0.17%, Table 3). These cells are indeed GFP− (arrowheads, FIG. 7C), and are likely non-expressing integrations of the GFP transgene.

TABLE 6
Per-cell confirmation of single cell PCR results by FACS. Results from 8
index-sorted plates are shown (FIGS. 7C-7D). For each assayed cell that was
successfully lysed and amplified (RNAseP+), the detection of PsiGag sequence was
compared to the expression of GFP from the LentiGFP transgene.

| | #cells | # | # | # cells | # | # |
|---|---|---|---|---|---|---|
| | identified as | transduced | transduced | identified as | untransduced | untransduced |
| Plate | transduced | GFP+ | GFP− | untransduced | GFP− | GFP+ |
| 1 | 34 | 32 | 2 | 52 | 51 | 1 |
| 2 | 32 | 31 | 1 | 49 | 46 | 3 |
| 3 | 12 | 12 | 0 | 76 | 75 | 1 |
| 4 | 22 | 21 | 1 | 65 | 58 | 7 |
| 5 | 47 | 45 | 2 | 43 | 39 | 4 |
| 6 | 8 | 7 | 1 | 81 | 80 | 1 |
| 7 | 18 | 16 | 2 | 64 | 61 | 3 |
| 8 | 43 | 41 | 2 | 44 | 43 | 1 |
| Total | 216 | 205 | 11 | 474 | 453 | 21 |
| | | (94.91%) | (5.09%) | | (95.57%) | (4.43%) |

### Comparison of TE in Single Cells and Methocult Colonies

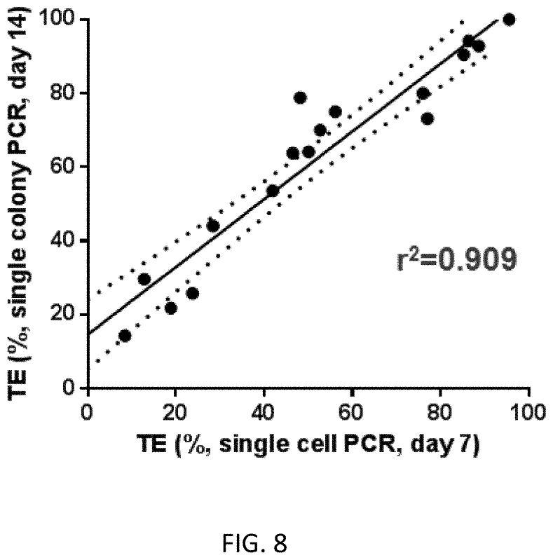

The single cell transduction efficiency assay provides a significantly faster and easier alternative to the traditional single colony TE assay. In the single colony assay, CD34+ cells are grown in Methocult for 14 days, and ˜25% of CD34+ cells form spatially-isolated colonies that are assumed to derive from individual cells. The genome of each colony forming cells is amplified as the cells divide to form colonies, obviating the need for PCR preamplification, and each colony is analyzed directly by qPCR for presence/absence of PsiGag and RNAseP sequences. To compare the single cell and single colony PCR assays, CD34+ cells were transduced with LentiGlobin BB305 lentiviral vector and grown either in SCGM growth media for 7 days or Methocult for 14 days. The TE in liquid culture was determined by single cell PCR and compared to percent transduced colonies (FIG. 8). High linear correlation was observed between the two assays (r²=0.909). The linear fit equation (slope=0.919, y-intercept=14.62) is consistent with higher marking in colony forming cells, as the majority of colony forming cells are erythroid, and erythroid progenitors are more transducible, whereas the IL-3 in SCGM growth media promotes outgrowth of less-transducible myeloid cells.

### Intra-Assay and Intermediate Precision

Transduced CD34+ cells were thawed and cultured in SCGM growth media for 4 days. Multiple vials of cells were then frozen so that replicates between days and analysts would be from the same sample. On 3 separate days, 3 vials were thawed by each Analyst. For each vial of cells, transduction efficiency was determined by single cell PCR. In the first analysis, each sample was analyzed using one 96-well plate, in triplicate (Table 7). Intra-assay precision was measured between two analysts and three days. In the second analysis (Table 8), TE was calculated using combined data points for three plates assayed each day, thereby reducing the statistical margin of error of the assay by tripling the number of single cell data points per sample. As expected, % CV decreased when each sample was analyzed using three 96-well PCR plates.

TABLE 7
Precision of measuring TE with the single cell PCR assay, one plate per sample.
Assay Variability (1 plate per sample 3 replicates/day, 3 days, 2 Analysts)

| Analyst 1 | | | | | | Analyst 2 | | | | | |
|---|---|---|---|---|---|---|---|---|---|---|---|
| Sample | Replicate | TE (%) | Average | StDev | % CV | Sample | Replicate | TE (%) | Average | StDev | % CV |
| Day 1 | 1 | 19.18 | 29.81 | 9.22 | 30.92 | Day 1 | 1 | 34.78 | 34.22 | 3.42 | 9.99 |
| | 2 | 34.62 | | | | | 2 | 30.56 | | | |
| | 3 | 35.62 | | | | | 3 | 37.33 | | | |
| Day 2 | 1 | 33.77 | 36.27 | 3.92 | 10.81 | Day 2 | 1 | 34.52 | 37.91 | 3.90 | 10.28 |
| | 2 | 34.25 | | | | | 2 | 37.04 | | | |
| | 3 | 40.79 | | | | | 3 | 42.17 | | | |
| Day 3 | 1 | 31.25 | 37.13 | 5.14 | 13.85 | Day 3 | 1 | 42.17 | 35.80 | 7.46 | 20.85 |
| | 2 | 39.34 | | | | | 2 | 27.59 | | | |
| | 3 | 40.79 | | | | | 3 | 37.65 | | | |

Day to Day Variability n = 9 for each Analyst)

| Analyst 1 | | | Analyst 2 | | | Intermediate Variability (Analyst 1 to Analyst 2, n = 18) | | |
|---|---|---|---|---|---|---|---|---|
| Average | StDev | % CV | Average | StDev | % CV | Average | StDev | % CV |
| 34.40 | 6.61 | 19.22 | 35.98 | 4.82 | 13.39 | 35.19 | 5.67 | 16.11 |

TABLE 8
Precision of measuring TE with single cell PCR assay, three plates per sample.
Assay Variability (3 plates per sample, 3 days, 2 Analysts)

| Analyst 1 | | | | | Analyst 2 | | | | |
|---|---|---|---|---|---|---|---|---|---|
| | TE (%) | Average | StDev | % CV | | TE (%) | Average | StDev | % CV |
| Day 1 | 29.91 | 34.50 | 4.01 | 11.62 | Day 1 | 34.26 | 35.95 | 1.84 | 5.11 |
| Day 2 | 36.28 | | | | Day 2 | 37.90 | | | |
| Day 3 | 37.31 | | | | Day 3 | 35.69 | | | |

Intermediate Variability (Analyst 1 to Analyst 2, n = 6)

| Average | StDev | % CV |
|---|---|---|
| 35.23 | 2.90 | 8.23 |

### Assay Linearity and Range

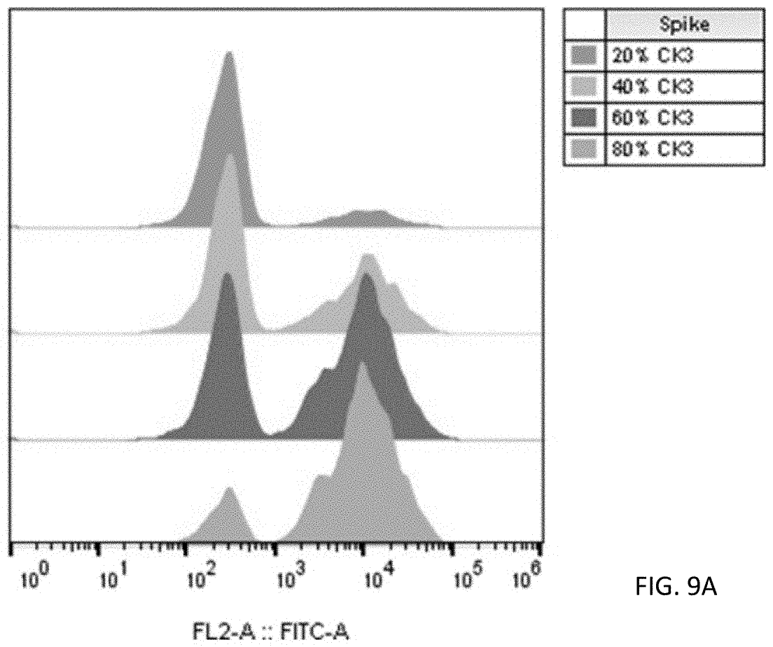

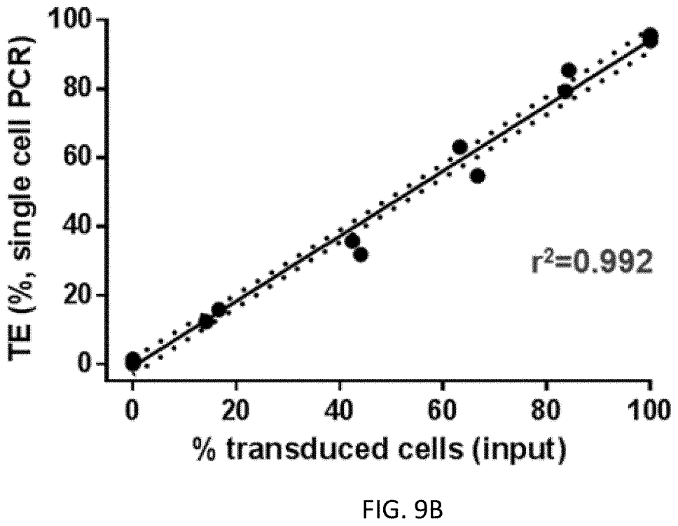

Linearity was evaluated by mixing CK3 cells with untransduced CD34+ cells at 20%, 40%, 60%, and 80% of the total cell population. The exact abundance of CK3 cells in the spike was determined by FACS, as CK3 cells are GFP+ whereas CD34+ cells are not (FIG. 9A). Data from unmixed CK3 and CD34+ cells was used for the 0 and 100% data points. Linear regression demonstrated that the assay was linear over the range of 0% to 100% TE (FIG. 9B, r²=0.992).

### Assay Performance Metrics and Acceptance Criteria

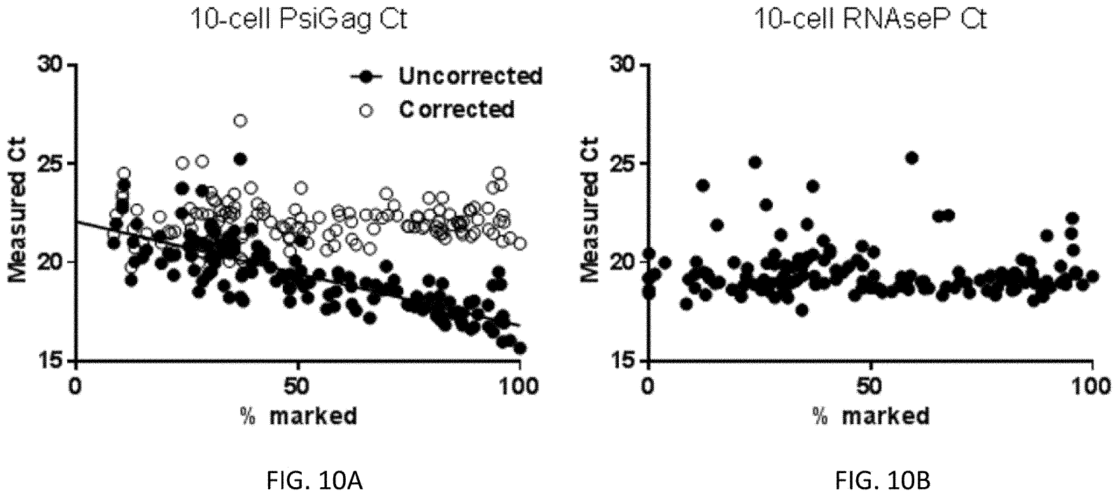

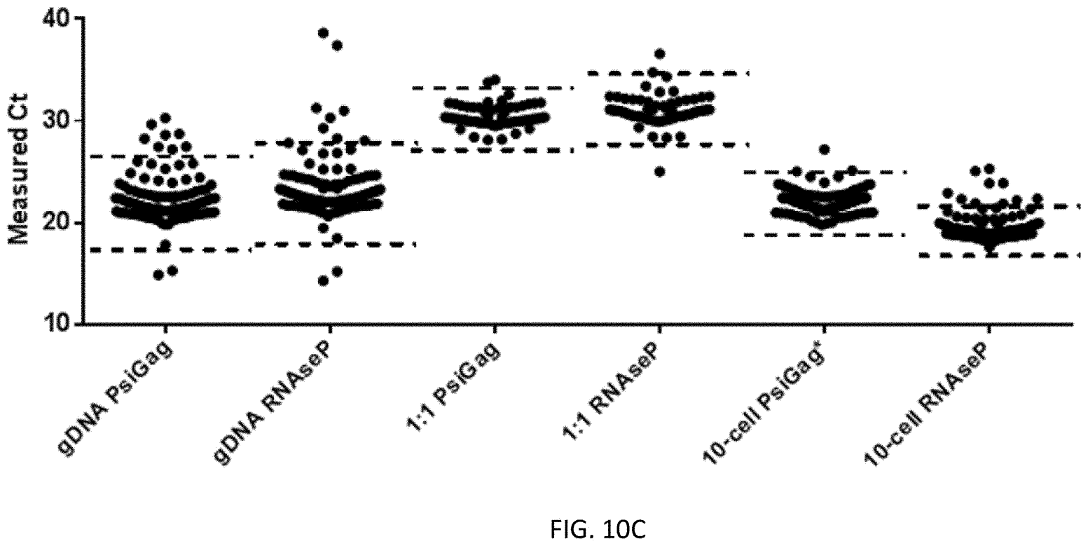

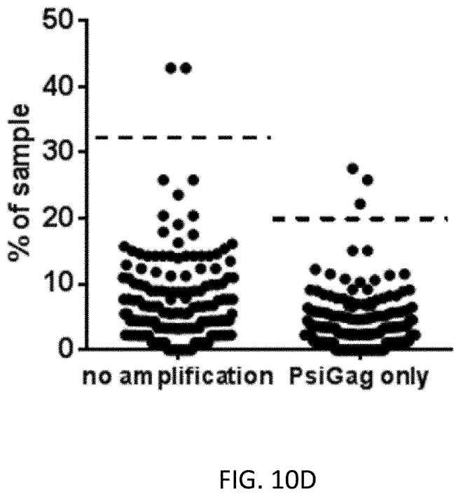

Retrospective analysis of assay acceptance controls and sample acceptance controls (FIG. 10, Table 9) was used to determine assay acceptance criteria (Table 10). Assay acceptance controls utilize a known amount of two standards: (1) 18 pg (5 genome equivalents) of CK3 gDNA, added to each 96-well plate prior to PCR pre-amplification, and (2) 1:1000 diluted plasmid added to each 384-well plate prior to qPCR. For plates with replicates of assay acceptance controls, the analyzed data is the average of the replicates. Sample acceptance controls utilize the tested sample to control for assay performance. Ten cells from each sample are deposited into three replicate wells of the 96-well plate during single-cell sorting, controlling for efficiency of cell deposition, cell lysis, and both PCR and qPCR reactions. Ideally, all single cell sample wells should be positive for RNAseP, and the percentage of sample wells with no amplification for either RNAseP or PsiGag is determined, measuring the efficiency of single cell deposition and lysis. All single cell sample wells positive for viral PsiGag should also be positive for genomic RNAseP, and the percentage of single cell readouts positive only for PsiGag but negative for RNAseP reflects either problems with multiplexed PCR/qPCR reactions or possible well contamination with extracellular DNA.

The 10-cell PsiGag Ct was found to be linearly correlated with the observed percentage of marked cells in the tested sample (FIG. 10A, Pearson coefficient −0.80, two-tailed p value<0.0001), whereas the 10-cell RNAseP Ct had no correlation with percent marked (FIG. 10B, Pearson coefficient −0.06, two-tailed p value 0.44). From the linear fit of the 10-cell PsiGag Ct vs TE, the slope was found to be −0.053, and the observed 10-cell PsiGag Ct was adjusted using the following equation:

Adjusted Ct=Ct+0.053*% TE Equation 7

Raw Ct data (FIG. 10C) was filtered for potential outliers using the ROUT method in GraphPad Prism. The false discovery rate for outliers was set to 0.1%. Outlier identification using the quartile method or the individual and moving range control chart produced similar results. The resulting data set was used to calculate the mean, standard deviation, and percent CV (Table 10). Upper and lower acceptance limits were set at three standard deviations from the mean. The data for percent no amplification and percent PsiGag only did not have a normal distribution (FIG. 10D), and correction by square root transformation followed by ROUT outlier filtering was used to calculate upper acceptance limit using the square root transformed mean and standard deviation (Table 10).

TABLE 9
Description of assay acceptance controls and sample acceptance controls.

| Assay control | Control type | Control for |
|---|---|---|
| 18 pg CK3 gDNA PsiGag Ct | Assay acceptance control | Efficiency of nested PsiGag |
| | | PCR/qPCR |
| 18 pg CK3 gDNA RNAseP Ct | Assay acceptance control | Efficiency of nested RNAseP |
| | | PCR/qPCR |
| 1:1000 p305 PsiGag Ct | Assay acceptance control | Efficiency of PsiGag qPCR |
| 1:1000 p305 RNAseP Ct | Assay acceptance control | Efficiency of RNAseP qPCR |
| 10-cell PsiGag Ct | Sample acceptance control | Efficiency of cell lysis and |
| | | PsiGag nested PCR/qPCR |
| 10-cell RNAseP Ct | Sample acceptance control | Efficiency of cell lysis and |
| | | RNAseP nested PCR/qPCR |
| % of wells with no | Sample acceptance control | Efficiency of single cell |
| amplification | | deposition and cell lysis |
| % of wells with PsiGag | Sample acceptance control | Efficiency of multiplexed |
| amplification but not RNAseP | | single cell PCR |

TABLE 10
Performance of assay acceptance controls and sample acceptance controls.
Limits are calculated as Mean ± 3 StDev. Assay acceptance criteria is based on the
calculated upper and lower limits for each control.

| | Data | Data | | | | Upper | Lower |
|---|---|---|---|---|---|---|---|
| Assay control | transformation | set size | Mean | StDev | % CV | limit | limit |
| 18pg CK3 gDNA PsiGag | none | 110 | 21.94 | 1.41 | 6.44 | 26.18 | 17.70 |
| Ct | | | | | | | |
| 18pg CK3 gDNA RNAseP | none | 114 | 22.97 | 1.67 | 7.27 | 27.98 | 17.96 |
| Ct | | | | | | | |
| 1:1000 p305 PsiGag Ct | none | 71 | 30.45 | 1.07 | 3.51 | 33.66 | 27.24 |
| 1:1000 p305 RNAseP Ct | none | 69 | 31.03 | 1.15 | 3.72 | 34.49 | 27.57 |
| 10-cell PsiGag Ct | Ct + 0.053 * % TE | 141 | 22.04 | 0.98 | 4.43 | 24.97 | 19.11 |
| 10-cell RNAseP Ct | none | 145 | 19.26 | 0.77 | 4.00 | 21.57 | 16.95 |
| % of wells with no | square root | 152 | 6.00 | 6.53 | 108.83 | 32.60 | — |
| amplification | | | | | | | |
| % of wells with PsiGag | square root | 151 | 3.03 | 4.37 | 144.22 | 21.79 | — |
| amplification but not | | | | | | | |
| RNAseP | | | | | | | |

### Comparison of TE in HSCs vs All Cells

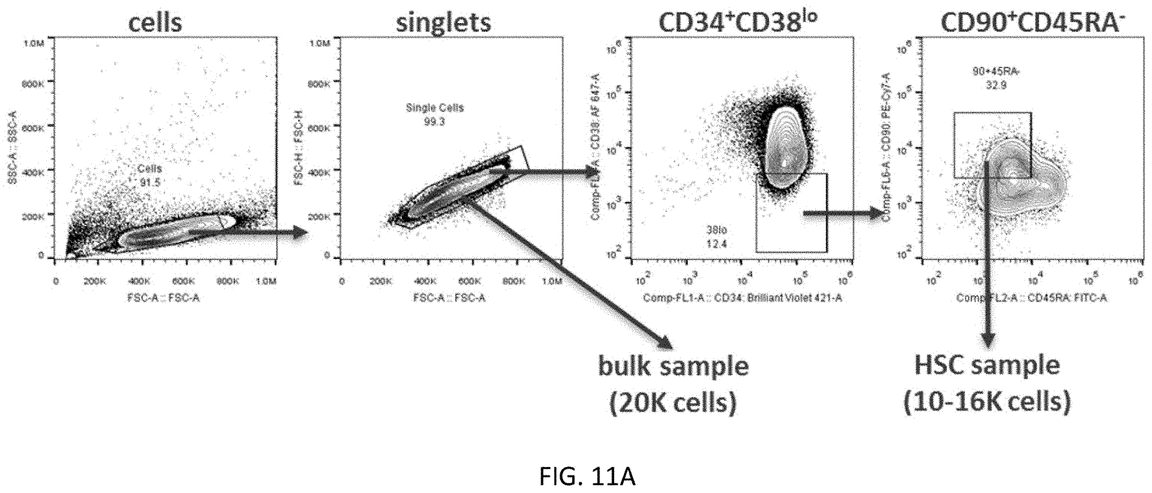

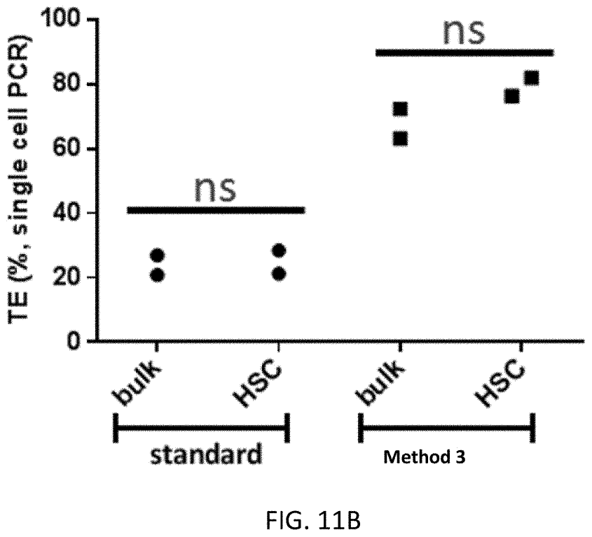

Because the single cell marking assay requires less than five thousand cells, it becomes possible to evaluate marking in rare subpopulations, such as hematopoietic stem cells (HSCs). To illustrate this approach, CD34+ cells were transduced with LentiGlobin BB305 LVV using standard (protamine sulfate) conditions or other transduction methods. Following post-transduction, cells were washed, stained with CD34+-BV421, CD38-AF647, CD90-PeCy7, and CD45RA-FITC, and either 20,000 singlets or 10,000-16,000 phenotypic HSCs were FACS-sorted from each sample. Sorted cells were cultured for 4 days in 100 μL SCGM growth media, followed by single cell PCR analysis (FIG. 11A). The difference in TE in HSCs vs bulk cells was not statistically significant (FIG. 11B, unpaired student's t-test p≥0.168).

### Comparison to Bulk VCN Readouts at Day 7 and Day 14

In the case of LentiGlobin, transduction efficiency has traditionally been estimated from the vector copy number, taken as an average measurement from the DNA pooled from thousands of cells. If the frequency of viral integrations per cell is random, the relationship between vector copy number (VCN) and transduction efficiency (TE) is a Poisson distribution (Equations 4, 5):

$$VCN = - 1 * ln ( 1 - ( \frac{TE}{100} ) ) \qquad ( Equation 4 )$$

$$TE ( \% ) = 100 * ( 1 - {e}^{- 1 * VCN} ) \qquad ( Equation 5 )$$

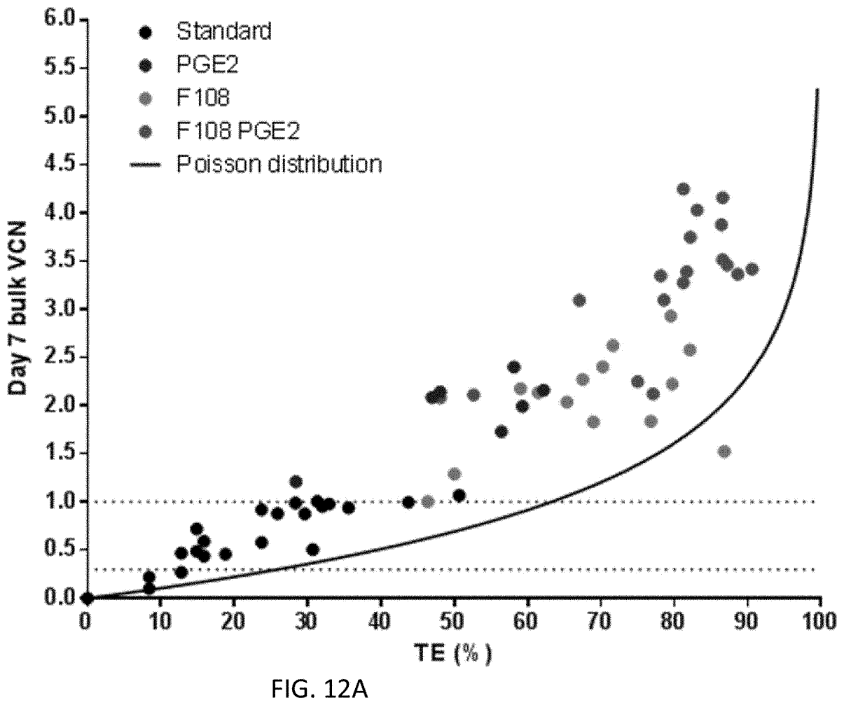

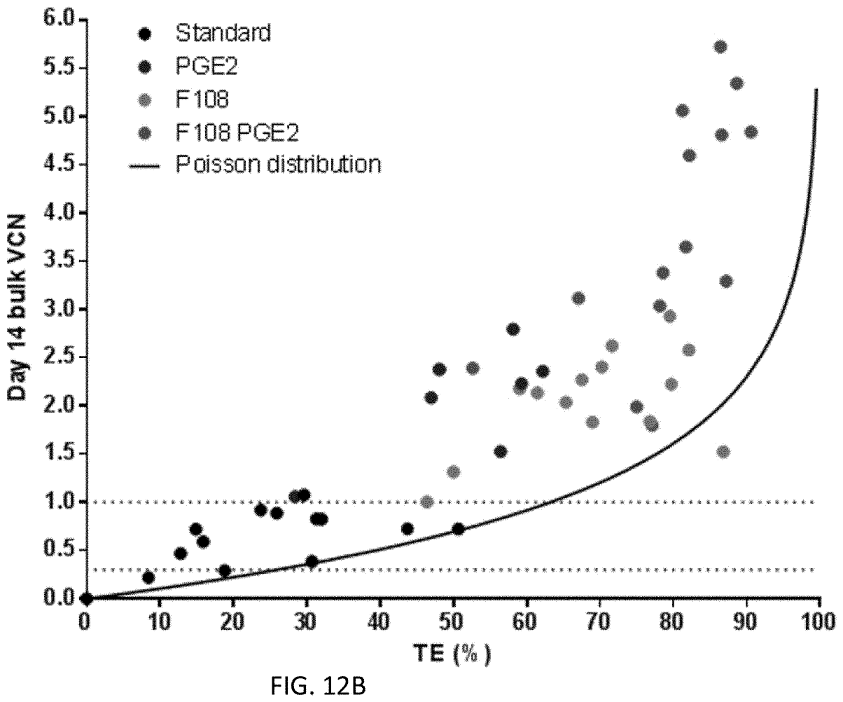

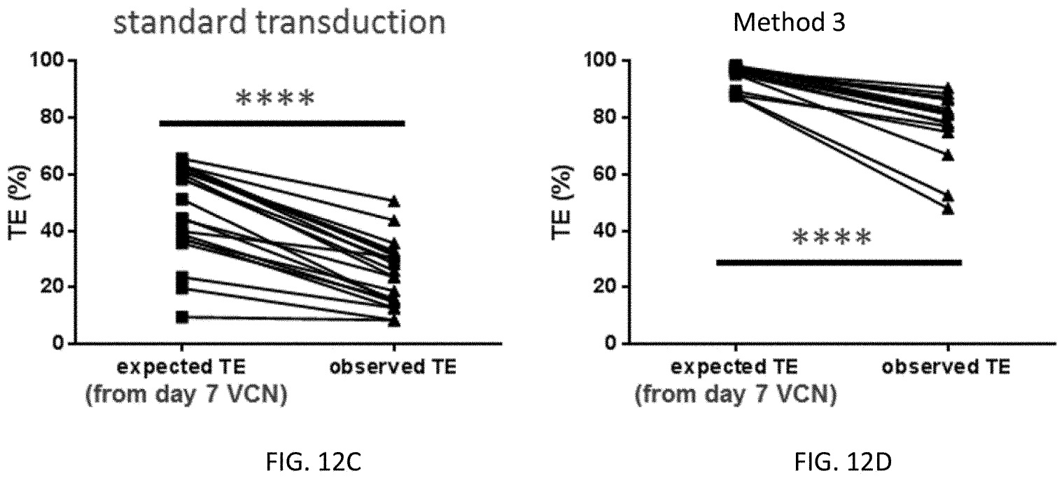

To directly compare VCN and TE, transduced CD34+ cells were cultured for seven days in SCGM growth media and analyzed both by single cell PCR assay and by traditional pooled VCN assay (FIG. 12A, n=61). In addition, pooled colony VCN was obtained from parallel 14-day cultures in Methocult (FIG. 12B, n=50). In both cases, large deviations from Poisson distribution were observed. For example, at a VCN=1, Equation 5 predicts 63% TE, whereas the observed transduction efficiency was 25-50%. The observed difference between expected and measured TE is statistically significant (p<0.0001), with a mean difference of 23% for standard transduction and 17% when using another transduction method (FIGS. 12C-12D). These differences are larger than the 5% false negative rate or the 11% margin of error of the assay, indicating that frequency of LVV integrations does not follow a random distribution. The nearly 2-fold spread in the observed TE at VCN=1 is much larger than the precision of the assay (average % CV=8.3, maximum % CV=30.9). Experiments are ongoing to understand the source of the large variation in TE at any given VCN.

### Identification of Potential Outliers by Paired Measurements of Single Cell TE and Bulk VCN

During evaluation of assay precision (Table 7), a sample with a bulk VCN of 0.98 was evaluated 18 times by single cell PCR. Although the average transduction efficiency was 35.2% and intermediate CV was 16.11%, one of the measurements (Analyst 1, Day 1, Replicate 1) had a TE of 19.2%. The 18 data points from the precision study were combined with 22 data points from standard transduction of CD34+ cells. The combined data set was fitted with a Poisson equation modified with an unconstrained constant k (Equation 6):

$$VCN = - \frac{1}{k} ln ( 1 - ( \frac{TE}{100} ) ) \qquad ( Equation 6 )$$

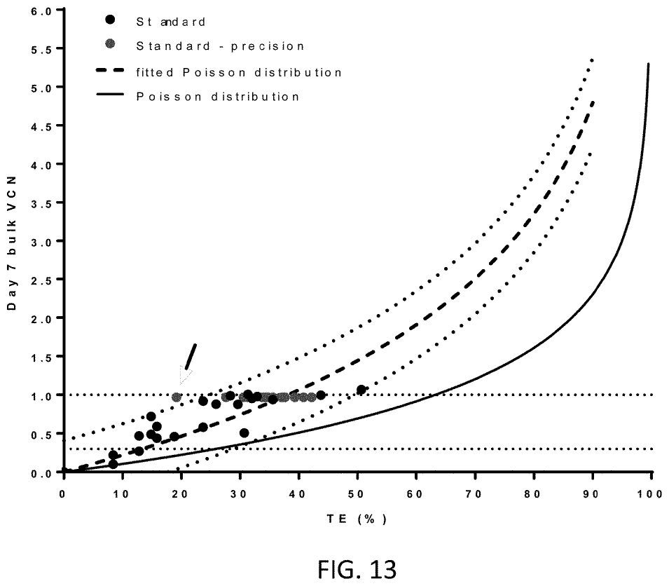

The data point 19.2% marked at VCN 0.98 was outside the 95% prediction interval of the non-linear fit with Equation 6 (FIG. 13), and is therefore likely an outlier. Paired vector copy number and transduction efficiency measurements therefore give further confidence to the TE determined by single cell PCR using a single 96-well plate per sample. Overall, for critical samples, each sample may be analyzed with three 96-well plates to reduce margin of error and increase precision. For other studies, a single 96-well plate is sufficient and comparison with bulk VCN can identify potential outlier measurements and justify re-testing.

### Conclusions

A single cell PCR assay was developed and qualified for measuring the percentage of cells transduced with a lentiviral vector (e.g., LentiGlobin BB305 lentiviral vector). Using stably transduced CK3 cell line, the assay was shown to have a 5.02% false negative rate. Transduction of CD34+ cells with LentiGFP followed by analysis of GFP+ cells produced a similar false negative rate of 4.43%. In untransduced CD34+ cells, the assay was shown to have a 0.17% false positive rate. Accuracy was confirmed by transducing cells with LentiGFP LVV and comparing the percent PsiGag positive by single cell PCR to percent GFP positive by FACS (r²=0.960) and correlation with index FACS sorting. The assay is therefore sufficiently sensitive and specific for accurate analysis of single cell genomes.

Following transduction, non-integrated LVV was found to decay in 3 days, with consistent assay readout between 3 and 7 days following transduction. The stabilization in TE is faster than the stabilization in bulk VCN, which has been shown to take 4-5 days, presumably because transduced cells have more non-integrated pro-viral DNA. Freezing of cells prior to analysis had no significant effect on results, permitting flexibility between drug product manufacturing and sampling for analysis of TE. The assay can be run with as few as 5,000 transduced cells, allowing for measurement of TE in rare cell types, such as HSCs. The assay is linear both by spike-in experiments (range: 0-100%, r²=0.992), and by transduction of CD34+ cells with increasing amount of LentiGFP (range: 12-73%, r²=0.960). Regardless of the linear fit used, the assay readout slightly under-represents actual TE in the sample, consistent with the measured false-negative rate. In addition, because this assay uses a limited set of single cell data points to extrapolate TE for the entire sample, the statistical margin of error can be calculated simply based on the measured TE, the number of data points sampled, and the confidence interval.

In cases where clinical decisions need to be made based on a minimum desired transduction efficiency for the drug product, Table 11 can be used to adjust the TE by the false negative rate and provide the margin of error of the measured TE within a 95% confidence interval. For example, the measurement of 35% marked cells in the single plate precision experiments corresponds to actual TE of 37±10% (Table 11). The observed standard deviation among 18 assay replicates was 5.67%, corresponding to the 95% confidence interval of ±11%, consistent with the predicted margin of error of ±10%. If three 96 well plates are used to measure the sample, as should be the case for clinical samples, the predicted TE becomes 37±6% (Table 11), consistent with the standard deviation among 6 assay replicates of 2.90%, corresponding to the 95% confidence interval of ±5.6%. The assay precision therefore is consistent with the statistical margin of error, that the assay is suitable for measurement of percent transduced cells with the margin of error indicated in Table 11. Because the assay margin of error can be reduced by running multiple assay plates per sample, the choice for the number of plates per sample should depend on the balance between assay throughput and desired readout accuracy.

During the measurement of assay precision in single 96-well plates, one of the 18 replicate results produced a value of 19.18%, outside the 95% confidence interval of 35±9%. One strategy to remove potential outlier values is to measure both bulk sample VCN and single cell TE. Although the observed transduction efficiencies do not follow the Poisson distribution, fitting the Poisson equation to the data produces a 95% prediction interval that predicts that 19.18% TE at a VCN of 0.98 is an outlier and would justify re-testing. Alternatively, increasing the number of assay plates per sample would also decrease the likelihood of an outlier measurement. Paired TE and VCN measurements could be performed at least after 4 days in culture, as pro-viral DNA takes longer to decay in transduced cells, impacting the bulk VCN readout.

Assay controls were developed and acceptance criteria determined by retrospective analysis of over 69 assay replicates. The average Ct values from each of the three control types must be within the limits listed in Table 10. In our approach, at most one replicate of the controls can be excluded from the calculation. The maximum number of RNAseP− single-cell wells must be below the limit indicated in Table 10, and no PsiGag or RNAseP amplification should be observed in the empty wells.

TABLE 6
Measured TE as an estimate of actual TE in the sample. Measured
TE is adjusted using the average of the slope and intercept obtained
by linear fits of spiked CK3 cells and LentiGFP data. Margin of
error within a 95% confidence interval is calculated based on the
measured TE obtained from 80 (1 plate) and 240 (3 plates) single
cell data points.

| TE | corrected TE, MOE from | corrected TE, MOE from |
|---|---|---|
| (measured) | 1 × 96-well plate per sample | 3 × 96-well plates per sample |
| 5 | 8 +/− 5% | 8 +/− 3% |
| 10 | 12 +/− 7% | 12 +/− 4% |
| 15 | 17 +/− 8% | 17 +/− 5% |
| 20 | 22 +/− 9% | 22 +/− 5% |
| 25 | 27 +/− 9% | 27 +/− 5% |
| 30 | 32 +/− 10% | 32 +/− 6% |
| 35 | 37 +/− 10% | 37 +/− 6% |
| 40 | 42 +/− 11% | 42 +/− 6% |
| 45 | 47 +/− 11% | 47 +/− 6% |
| 50 | 52 +/− 11% | 52 +/− 6% |
| 55 | 57 +/− 11% | 57 +/− 6% |
| 60 | 62 +/− 11% | 62 +/− 6% |
| 65 | 67 +/− 10% | 67 +/− 6% |
| 70 | 72 +/− 10% | 72 +/− 6% |
| 75 | 77 +/− 9% | 77 +/− 5% |
| 80 | 82 +/− 9% | 82 +/− 5% |
| 85 | 87 +/− 8% | 87 +/− 5% |
| 90 | 92 +/− 7% | 92 +/− 4% |
| 95 | 97 +/− 5% | 97 +/− 3% |
| 99 | 101 +/− 2% | 101 +/− 1% |

Prior to the development of this assay, transduction efficiency of LentiGlobin could only be measured by single colony PCR or estimated from bulk VCN using the Poisson equation. The single colony and single cell PCR assays have a similar margin of error, generally measuring <100 colonies or single cells per sample. Measuring TE in single cells pooled from Methocult colonies is not accurate because colonies have a vastly different size distribution and therefore larger colonies are over-represented. Instead, the TE observed at day 7 in SCGM growth media was compared to the TE observed in Methocult colonies, and a linear correlation was observed between the two assays (r²=0.909). The TE was generally higher in Methocult colonies, as expected, because erythroid colony forming cells are generally more permissive to transduction and produce the majority of colonies in Methocult.

The measured transduction efficiency greatly deviated from that estimated from bulk VCN by Poisson distribution. For example, at VCN=1, it is predicted that 63% of the cells are transduced. In reality, at VCN=1 in either liquid culture or pooled Methocult colonies corresponds to 25-50% TE (FIG. 12). The observed mean difference was 23.2% for standard transductions (FIG. 12C, paired t-test p-value of <0.0001), a deviation much greater than the 11% margin of error in the assay (Table 11) or error in assay precision. Estimating TE from VCN using a Poisson distribution therefore gives a large over-estimation of the true TE in the sample and should be avoided. Instead, TE should be measured directly, and this assay is suitable for measuring the percentage of cells transduced with LentiGlobin.

### Methods

### Study Design

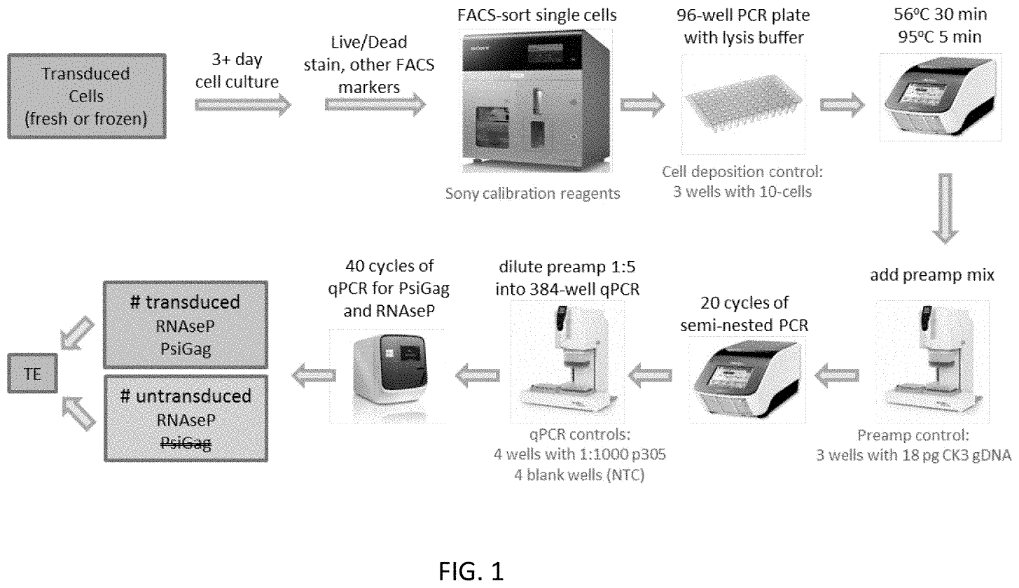

The schematic outline of the single cell PCR assay is shown in FIG. 1. A number of PsiGag and RNAseP primers and probes and PCR conditions were evaluated until sufficient single cell sensitivity was achieved in CK3 cells. Specificity of PsiGag amplification was confirmed in untransduced CD34+ cells. The number of 96-well plates needed to characterize transduction efficiency in a sample was determined using statistical margin of error formula. The minimum time in culture required after transduction to degrade the non-integrated viral DNA was determined, and effects of sample cryopreservation prior to analysis was evaluated. Accuracy was confirmed by transducing CD34+ cells will LentiGFP and comparing the percentage of cells that are PsiGag+ by single cell PCR to the percentage of cells that are GFP+ by FACS and index sorting. The percentage of PsiGag+ cells by single cell PCR was also compared to the percentage of PsiGag+ colony forming cells. Assay precision was evaluated by thawing aliquots of transduced CD34+ cells and repeating the assay in triplicate on three separate days with two operators. Linearity was demonstrated by spiking a known percentage of CK3 cells into un-transduced CD34+ cells and measuring the percentage of PsiGag+ CK3 cells by single cell PCR. Retrospective analysis of assay acceptance controls and sample acceptance controls was used to establish assay acceptance criteria. The single cell PCR assay was then used to specifically measure TE in HSCs, and to compare TE to VCN at day 7 in liquid culture and day 14 in Methocult. Finally, the use of paired measurements of VCN from pooled cells and TE from single cells was investigated as a method to confirm the accuracy of measuring TE.

### Single Cell FACS-Sorting

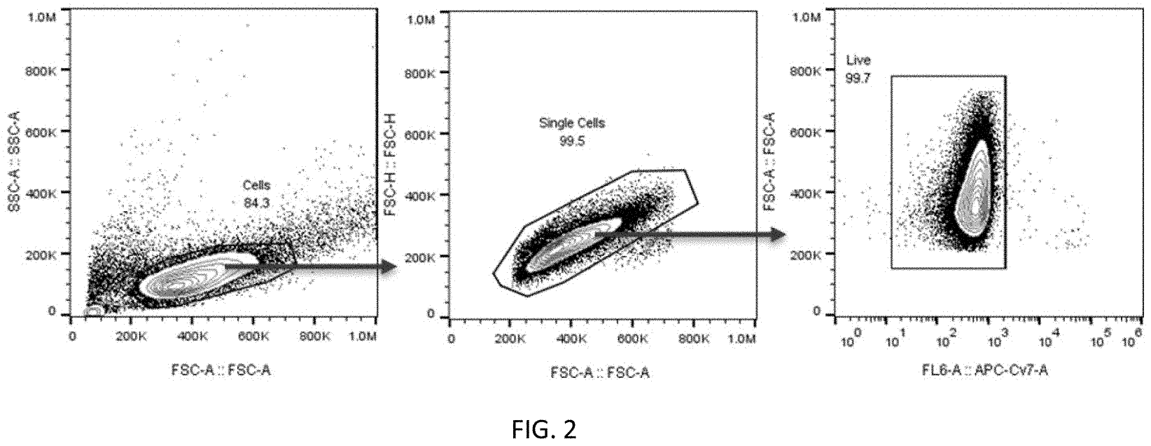

If samples were frozen, cells were thawed in SCGM with DNAseI. For cell staining, CK3 and/or CD34+ cells were collected in a 1.7 mL tube and centrifuged at 500×g for 5 minutes to obtain a cell pellet. The media was carefully aspirated from the tube and cells were reconstituted in 200-600 μL of a 1:1000 dilution of LIVE/DEAD™ Fixable Near-IR Dead Cell Stain. The cells were incubated at room temperature for 10-30 minutes and then centrifuged at 500×g for 5 minutes. The media was carefully aspirated from the tubes and cells were reconstituted in 200-600 μL of FACS Buffer (2% HABS+PBS). The cell suspension was then passed through a cell strainer into a FACS tube (if volume was >300 μL) or a microtiter tube (if volume was <300 μL). Cells were separated from debris using a FSC-Area/SSC-Area gate, gated on singlets with a FSC-Area/FSC-Height gate, and gated on viable cells with an APCCy7/SSC-Area gate (FIG. 2). Viable single cells were sorted into a 96 well PCR plate with each well containing 10 μL of lysis buffer (Water, 1×Taq Buffer with KCl, 0.1 mg/mL Proteinase K). Plates were sealed with film, vortexed to get the cell into solution, and centrifuged.

### Cell Lysis and Preamplification

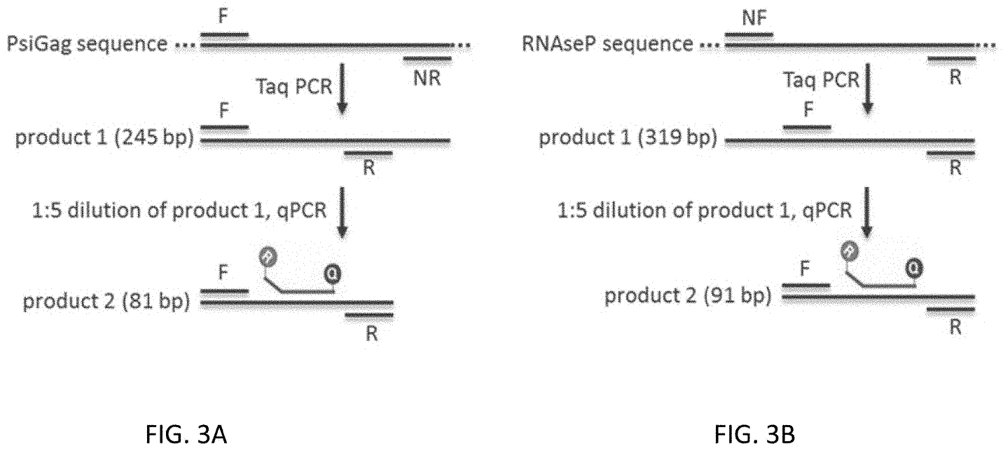

96 well PCR plates containing single cells in lysis buffer were placed in a thermocycler at 56° C. for 30 minutes followed by 5 minutes at 95° C. After lysis, the plates were held at 4° C. Preparation of the preamplification master mix was done during the lysis cycle and placed on ice until needed. Preamplification master mix consisted of lx PCR Gold Buffer, 2 mM MgCl₂, 0.5 mM dNTPs, 0.36 μM of each preamplification primer (PsiGag-F, PsiGag-NR, RNAseP-NF, RNAseP-R, FIG. 3), and 2.5 U AmpliTaq in water. When lysis was completed, plates were removed from the thermocycler, unsealed, and 15 μL of preamplification was added to each well using the VIAFLO, for a total volume per well of 25 μL. Pipette tips were changed in between each sample to prevent cross contamination. Plates were securely sealed, vortexed, and centrifuged. Plates were placed on a thermocycler for 10 minutes at 95° C., followed by 20 cycles: 15 seconds at 95° C., 30 seconds at 55° C., 1 minute at 72° C. When cycles were completed, plates were held at 4° C.

### qPCR and Data Analysis

When preamplification was completed, plates were removed from the thermocycler and carefully unsealed. Two microliters of preamplification material was removed from each the 96 well plates and placed in the wells of a 384 well plate that contained qPCR master mix using the VIAFLO. Master mix for qPCR consisted of 1×TaqMan, 0.7 μM of each primer (PsiGag-F, PsiGag-R, RNAseP-F, RNAseP-R, FIG. 3), 0.14 μM of each probe (PsiGag FAM, RNAseP VIC), and water. Eight microliters of qPCR master mix was added to each well of a 384 well using the VIAFLO. Plates were carefully sealed, vortexed, and centrifuged. The 384 well plates were placed in the StepOne Thermocycler for 2 minutes at 50° C., 10 minutes at 90° C., then 40 cycles of: 15 seconds at 95° C., 1 minute at 60° C. Data was analyzed at the conclusion of the qPCR after setting the threshold for PsiGag and RNAseP at 0.2. Data was then exported to Excel where it was further analyzed and separated into categories: number marked, number unmarked, number with no amplification, and number with only PsiGag amplification (Table 2). The cutoff Ct value for successful RNAseP and PsiGag amplifications was determined to be 32. The TE was calculated by dividing the number marked by the total of marked and unmarked cells, then multiplying by 100 (Equation 2). The percent of wells with no amplification was calculated as a percentage of all assayed single cell wells. The percent of wells with only PsiGag amplification was calculated as a percentage of all wells except those with no amplification.

### Single Colony Marking Analysis

Single colonies were picked from Methocult plates under a microscope and deposited in TaqMan Sample to SNP lysis buffer (20 μL), incubated for at least 3 minutes, followed by addition of TaqMan Sample to SNP stabilization buffer (20 μL). Each 20 μL qPCR reaction consisted of 1×GTXpress TaqMan, 1×RNAseP VIC copy number reference assay kit, 0.9 μM of each primer (PsiGag-F, PsiGag-R), 0.2 μM of PsiGag FAM probe, 4 μL of extracted single-colony DNA, and water. DNA from each colony was analyzed in triplicate in 96-well plates. CK3 gDNA was used in triplicate as a reference with VCN=2. qPCR was performed in the StepOne™ Thermocycler for 2 minutes at 50° C., 10 minutes at 90° C., then 40 cycles of: 15 seconds at 95° C., 1 minute at 60° C. Data was analyzed at the conclusion of the qPCR after setting the threshold for PsiGag and RNAseP at 0.2. Data was then exported into Excel, and VCN per colony was calculated relative to CK3. The RNAseP Cts were subtracted from the PsiGag Cts for all samples to calculate the ΔCt. The ΔCt for CK3 was subtracted from all samples to calculate the ΔΔCt, and VCN was calculated using Equation 1:

VCN=2*2^{-ΔΔCt} (Equation 1)

VCN values from triplicate qPCR reactions on each colony were averaged and rounded to a whole number. Any values with VCN<0.5 were considered unmarked, and any colonies with RNAseP Ct>32 were excluded. Under assay acceptance criteria, VCN for all CK3 replicates was 1.6-2.4 and non-targeting control wells had no qPCR amplification above the threshold.

### Primers and Probes

PsiGag-F (universal forward primer) 5′-ggagctagaacgattcgcagtta-3′ (SEQ ID NO: 1);

PsiGag-NR (nested reverse primer) 5′-cagctgctgcttgctgtgc-3′ (SEQ ID NO: 2);

PsiGag-R (qPCR reverse primer) 5′-ggttgtagctgtcccagtatttgtc-3′ (SEQ ID NO: 3);

PsiGag FAM probe: 5′-(FAM)-acagccttctgatgtctctaaaaggccagg-(TAMRA)-3′ (SEQ ID NO: 4);

RNAseP-R (universal reverse primer) 5′-gtggtctgaattgggttatgagg-3′ (SEQ ID NO: 5);

RNAseP-NF (nested forward primer) 5′-ggagggaagctcatcagtgg-3′ (SEQ ID NO: 6);

RNAseP-F (qPCR forward primer) 5′-ggagcttggaacagactcacg-3′ (SEQ ID NO: 7); and

RNAseP VIC Probe: 5′-(MAXN)-acctcacct/ZEN/cagccattgaactcacttcg-(IABkFQ)-3′ (SEQ ID NO: 8).

### Example 2

### Development of Single Cell PCR Assay to Quantify the Transduction Efficiency of Lentiviral Vectors in Peripheral Blood

% LVV+ is defined as a percentage of cells with at least one integration of LentiGlobin BB305 lentiviral vector (LVV). A single cell polymerase chain reaction (scPCR) using genomic DNA is utilized to measure % LVV+ drug product cells prior to infusion. A drug product with a high % LVV+ measurement is indicative of high transduction efficiency and potentially efficacious gene therapy (Example 1).

Clinical readout for subjects post infusion includes total g/dL of the therapeutic protein HBB-T87Q (Hemoglobin beta with glutamine substitution at residue 87, encoded by LentiGlobin BB305) produced in the peripheral blood (PB). In addition to quantifying the amount of HBB-T87Q protein in circulation, it is desirable to measure the proportion of transduced, circulating erythroid cells that produce HBB-T87Q. Red blood cells that produce therapeutic HBB-T87Q cannot be distinguished from red blood cells producing endogenous beta-globin by a typical fluorescence activated cell sorting (FACS) assay due to the absence of an antibody that can distinguish between HBB-T87Q and endogenous beta-globin. An existing scPCR assay is not applicable to red blood cells as they are enucleated and lack genomic DNA.

In this study, the scPCR approach has been adapted to measure % LVV+ circulating lympho-myeloid peripheral blood mononuclear cells (PBMCs). Hematopoietic stem and progenitor cells repopulate the lymphoid, myeloid, and erythroid compartments. Therefore, measuring the % LVV+ lymphoid and myeloid cells in circulation reflects the % LVV+ stem and progenitor cells that contribute to hematopoiesis, and estimates the proportion of erythroid cells that produce HBB-T87Q.

Prior to infusion, patients undergo myeloablative conditioning to enhance drug product engraftment efficiency. This conditioning does not clear T cells, which can exist prior to and long after infusion. Circulating T cell levels are variable, and the pre-infusion T cells could negatively skew the measured engraftment of genetically modified stem and progenitor cells if all PBMCs are used to determine % LVV+, especially when measured recently after infusion. A flow panel utilizing CD3 as a T cell marker was used to gate T cells out of peripheral blood samples prior to measuring % LVV+ cells. Except for the first feasibility sample, only CD45+CD3− cells (non T cell PBMCs) from subject PB were analyzed to quantify % LVV+ cells, consistent with vector copy number measurements performed post-infusion.

### Results

### Accuracy, LOQ, and LOD

To evaluate false negative rate, CK3 cells were spiked into PB and DNA+ cells were analyzed. Index sort data was compiled from 33 FACS-sorted 96-well plates. Of 1,397 sorted GFP+/DNA+ cells (CK3 cells), 95.5% were LVV+, indicating a 4.5% false negative rate, consistent with that discussed in Example 1 for scPCR (Table 12). 81 DNA+/GFP− cells were identified as LVV+, consistent with 5.5% GFP silencing previously observed in CD34 cells transduced with GFP LVV.

TABLE 12
Index data of nucleated cells from PB sample spiked with CK3.
The sorted plates were gated on the DNA+/GFP+ population,
n = 1397.

| Category | # of cells |
|---|---|
| GFP+LVV+ | 1334 |
| GFP+LVV− | 63(4.5% false negative) |
| Total | 1397 |

To evaluate false positive rate, untransduced PB samples from healthy donors were single cell sorted and analyzed. Of 895 sorted DNA+ cells, 99.2% were LVV−, indicating a false positive rate of 0.8% (Table 13).

TABLE 13
Assay accuracy and false discovery rates. Single cell data set
was generated by sorting untransduced nucleated cells from
PB, n = 895. The calculated false positive rate is 0.8%.

| Category | # of cells |
|---|---|
| GFP−LVV+ | 7 (0.8% false positive) |
| GFP−LVV− | 888 |
| Total | 895 |

Accuracy was assessed for the CD45/CD34/CD3 gating strategy by staining PBMCs spiked with CK3 and sorting PBMCs from both the “T cell” and “Not T cell” populations, and CK3 cells from the CD34+ population. CD34+ sorted CK3 cells should be LVV+ due to LentiGFP integration, and PBMCs sorted from either “T cell” or “Not T cell” gates should be LVV−. Of 111 CD34+ sorted cells, 96.4% were LVV+, indicating a 3.6% false negative rate. 99.6% of PBMCs sorted from the T cell and Not T cell gates were LVV−, indicating a false positive rate of 0.4% (Table 14). These results are consistent with RPT-0345 for scPCR of primary cells, and with those reported above for cells stained with only Draq5.

TABLE 14
Assay accuracy and false discovery rate using the CD45+CD34+
and CD45+CD3− staining panel. Data set was generated by
sorting CK3 spiked PBMCs from indicated gates, n = 364.

| | | | | false discovery |
|---|---|---|---|---|
| Gate | LVV+ cells | LVV− cells | total cells | rate |
| CD34+ (CK3) | 107 | 4 | 111 | 3.6% |
| T cell, Not T cell | 1 | 252 | 253 | 0.4% |
| (PBMCs) | | | | |

Margin of error (MOE) was used to estimate the limit of quantification (LOQ) where the measured value's 95% confidence interval is greater than the false positive rate of the assay. For a single 96-well plate per sample (n=80 cells), the LOQ is 6.4% LVV+, as the subtracted corresponding MOE of ±5.6% remains above the 0.8% false positive rate. For three 96-well plates per sample (n=240 cells), the LOQ is 3% LVV+, as the subtracted MOE of ±2.2% remains above the 0.8% false positive rate.

Limit of Detection (LOD) was determined using untransduced PBMCs when running one plate per sample and three plates per sample by adding 3 standard deviations (SD) of each data set to the highest % LVV+ result. 14 single plates of untransduced PBMCs (n=14) yielded a LOD of 5.2% for a single plate (Table 15), while four sets of triplicate plates (n=4) yielded a LOD of 1.1% for three plates (Table 16).

TABLE 15
Limit of detection when assaying one plate per sample. LOD is calculated
by adding 3 SD to the largest % LVV+ result for each data set.
Individual plates

| | % |
|---|---|
| Sample | marked |
| 1 | 1.2 |
| 2 | 0.0 |
| 3 | 0.0 |
| 4 | 1.2 |
| 5 | 0.0 |
| 6 | 0.0 |
| 7 | 0.0 |
| 8 | 1.2 |
| 9 | 0.0 |
| 10 | 1.2 |
| 11 | 2.5 |
| 12 | 0.0 |
| 13 | 2.4 |
| 14 | 0.0 |
| SD | 0.92 |
| 3*SD | 2.76 |
| LOD | 5.2 |

TABLE 16
Limit of detection when assaying three plates per sample. LOD is
calculated by adding 3 SD to the largest %LVV+ result for each data set.
Triplicate plates

| Sample | % |
|---|---|
| set | marked |
| 1 | 0.40 |
| 2 | 0.00 |
| 3 | 0.00 |
| 4 | 0.41 |
| SD | 0.23 |
| 3*SD | 0.70 |
| LOD | 1.1 |

### Assay Linearity

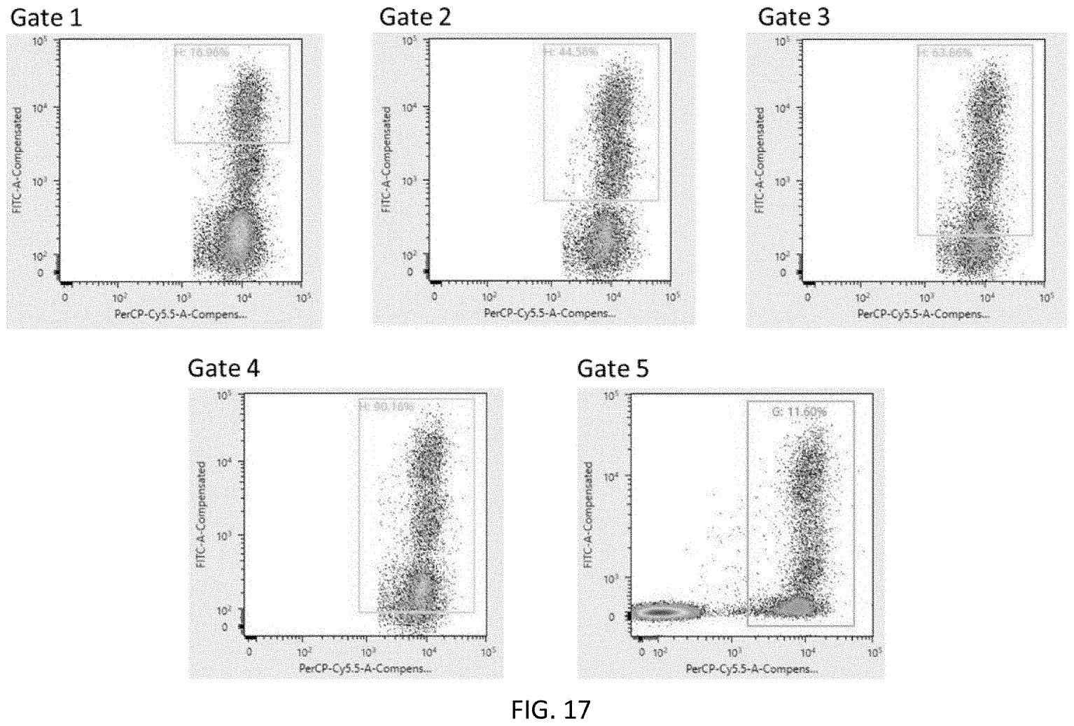

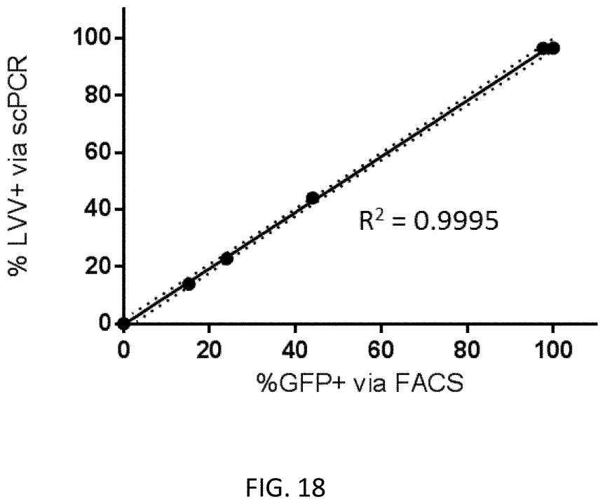

Assay linearity was evaluated by comparing the % LVV+ observed by scPCR to % GFP+ observed by FACS for healthy donor PB spiked with CK3. Five plates were sorted from five different gates that included increasing numbers of untransduced cells (FIGS. 17-18, linear fit r²=0.9995). The readout was linear (slope=0.98) and Y-intercept of −0.29 is within the measured 4.5% false negative rate.

### Assay Precision

HPD16020-Comp1 LentiGlobin transduced CD34+ cells and PBMCs were mixed and tested in triplicate on three separate days by two operators. Cells were sorted from the CD34+ gate to measure % LVV+HPD16020 cells within the PBMC matrix. Table 17 evaluates the precision of the assay when one 96-well plate is analyzed per sample, yielding a maximum % coefficient of variation (CV) of 6.8% between triplicates measured on the same day. Intermediate precision was evaluated for each analyst (n=9) with a maximum % CV of 4.9%, and between both analysts (n=18) with a maximum % CV of 4.4%.

Table 18 evaluates the precision of the assay when three 96-well plates are analyzed per sample, thereby reducing the statistical MOE of the assay (Example 1) by tripling the number of single cell data points per sample. As expected, % CV decreased when each sample was analyzed using three 96-well PCR plates. Consistent with Example 1, when high accuracy is desired, or when samples are thought to have a low % LVV+, three 96-well plates per sample should be run.

TABLE 17
Precision of measuring % LVV+ with PB scPCR, one plate per sample.
Assay Precision (1 plate per sample, 3 replicates/day, 3 days, 2 analysts)

| Analyst 1 | | | | | | Analyst 2 | | | | |
|---|---|---|---|---|---|---|---|---|---|---|
| Day | Replicate | % LVV+ | Avg. | SD | % CV | Replicate | % LVV+ | Avg. | SD | % CV |
| Day 1 | 1 | 83 | 84 | 1.4 | 1.7 | 1 | 90 | 86 | 3.5 | 4.0 |
| | 2 | 85 | | | | 2 | 83 | | | |
| | 3 | 85 | | | | 3 | 86 | | | |
| Day 2 | 1 | 84 | 86 | 4.5 | 5.3 | 1 | 80 | 82 | 1.8 | 2.2 |
| | 2 | 83 | | | | 2 | 84 | | | |
| | 3 | 91 | | | | 3 | 83 | | | |
| Day 3 | 1 | 89 | 84 | 4.9 | 5.9 | 1 | 81 | 87 | 6.0 | 6.8 |
| | 2 | 79 | | | | 2 | 90 | | | |
| | 3 | 84 | | | | 3 | 92 | | | |

| Day to Day Variability (n = 9 for each analyst) | | | | | | Intermediate precision | | |
|---|---|---|---|---|---|---|---|---|
| Analyst 1 | | | Analyst 2 | | | (analyst 1 to analyst 2, n = 18) | | |
| Average | SD | % CV | Avg. | SD | % CV | Average | SD | % CV |
| 85 | 3.5 | 4.2 | 85 | 4.2 | 4.9 | 85 | 3.8 | 4.4 |

TABLE 18
Precision of measuring % LVV+ with PB scPCR,
three plates per sample.
Assay Precision (3 plates per sample, 3 days, 2 Analysts)

| Analyst 1 | | | | | Analyst 2 | | | | |
|---|---|---|---|---|---|---|---|---|---|
| | % | | | % | | % | | | % |
| Day | LVV+ | Average | SD | CV | Day | LVV+ | Average | SD | CV |
| 1 | 84 | 85 | 1.0 | 1.2 | 1 | 86 | 85 | 2.6 | 3.0 |
| 2 | 86 | | | | 2 | 82 | | | |
| 3 | 84 | | | | 3 | 87 | | | |

Intermediate precision (analyst 1 to analyst 2, n = 6)

| Average | SD | % CV |
|---|---|---|
| 85 | 1.8 | 2.1 |

### Feasibility and % LVV+ Cells

Peripheral blood was collected from 9 subjects at various time points post infusion and processed. Between 0.5-1×10⁶ cells isolated by density gradient centrifugation were assayed either the day of receipt or following an overnight hold at 4C. % LVV+ was calculated from three 96-well plates of CD45+CD3− PB cells for 7 of the 9 samples, demonstrating assay feasibility (Table 19). Sample 1 PB was stained with Draq5 only and includes CD45+CD3+ T cells in the sort. Sample 9% LVV+ was calculated from a single 96-well plate for a stability study. % LVV+ infused drug product ranged between 20-92% (Table 20).

TABLE 19
% LVV+ PB as measured by PB scPCR in 9 samples.

| Subject | M4.5 | M9 | M15 | M21 |
|---|---|---|---|---|
| 1 | NA | NA | NA | 8%* |
| 2 | NA | NA | NA | 7% |
| 3 | NA | NA | NA | 8% |
| 4 | NA | NA | NA | 6% |
| 5 | NA | NA | 12% | NA |
| 6 | NA | NA | 6% | NA |
| 7 | 65% | NA | NA | NA |
| 8 | 21% | 23% | NA | NA |
| 9 | NA | 19% | NA | NA |

*Subject 1 PB was stained with Draq5 only and includes CD3+ T cells.

TABLE 20
% LVV+ infused drug product.

| | Drug |
|---|---|
| | Product |
| Subject | % LVV+ |
| 1 | 29 |
| 2 | 20 |
| 3 | 30 |
| 4 | 29 |
| 5 | 42 |
| 6 | 27 |
| 7 | 92 |
| 8 | 65 |

### Stability

To measure stability of PBMC composition, PB from a healthy donor was isolated by density gradient centrifugation and a CBC was performed on Day 0, 1, 2, and 3 post-blood draw to determine the relative abundance of different PB populations (Table 21). The lymphocyte population in isolated by the density gradient centrifugation PB remained stable at 2.9×10³ cells/μL. Monocytes gradually decreased from 0.4×10³ cells/μL to 0.2×10³ cells/μL and granulocytes gradually increased from 0.1×10³ cells/μL to 0.3×10³ cells/μL. Though the granulocyte and monocyte concentrations of the sample changed over the three days, the total white blood cell count remained stable at an average of 3.4×10³ cells/μL.

TABLE 21
Stability of lymphocytes, monocytes, and granulocytes isolated by density
gradient centrifugation from PB

| Days post- | Lymphocytes | Monocytes | Granulocytes | Total | % | % | % |
|---|---|---|---|---|---|---|---|
| blood draw | (×10³/μL) | (×10³/μL) | (×10³/μL) | WBC | Lymphocytes | Monocytes | Granulocytes |
| 0 | 2.9 | 0.4 | 0.1 | 3.4 | 85.7 | 12.3 | 2.0 |
| 1 | 2.9 | 0.3 | 0.2 | 3.4 | 85.5 | 8.1 | 6.4 |
| 2 | 2.7 | 0.2 | 0.3 | 3.2 | 84.2 | 6.3 | 9.5 |
| 3 | 3.1 | 0.2 | 0.3 | 3.6 | 85.2 | 6.2 | 8.6 |
| Average | 2.9 | 0.3 | 0.2 | 3.4 | 85.2 | 8.2 | 6.6 |

To measure the impact of sample stability on assay result, PB from Subject 9 was isolated by density gradient centrifugation after 48 hours in transit and PB scPCR was performed over three consecutive days to assess stability (Table 22). The sample was tested immediately after isolation by density gradient centrifugation (2 days post blood draw), and on the following two days (3 and 4 days post blood draw). One 96-well plate was tested each day, and the sample was stored at 4C in RPMI in-between testing. The measured % LVV+ remains stable 2 and 3 days post blood draw, but decreases 4 days post blood draw. Given that the measured % LVV+ cells, total WBC and lymphocyte population remain stable over 3 days and measured % LVV+ drops at day 4, samples must be tested within three days of blood draw.

TABLE 22
Stability of % LVV+ cells in PB sample. PB scPCR was performed on
three consecutive days. The sample was stored at 4 C. between days.

| Days post blood draw | % LVV+ | MOE |
|---|---|---|
| 2 days | 19 | ±8% |
| 3 days | 19 | ±8% |
| 4 days | 8 | ±6% |

### Conclusions

A scPCR assay was developed for measuring the percentage of nucleated cells transduced with LVV in PB. The assay can be applied to any lentiviral vector containing the PsiGag sequence (LentiD, LentiG, bb2121, LentiGFP, etc.) Using a stably transduced CK3 clonal cell line, the assay was shown to have a 4.5% false negative rate. In untransduced DNA+ cells from PB, the assay was shown to have a 0.8% false positive rate. Accuracy was confirmed by spiking PB with CK3 and comparing the proportion of PsiGag positive cells determined by scPCR to the proportion of GFP positive cells determined by FACS. Accuracy was not affected by incorporating CD45, CD34 and CD3 antibodies to sort CD45+CD3− PBMCs. The assay is therefore sufficiently sensitive and specific for accurate analysis of single cell genomes.

The assay was found to be linear in the range of 0 to 96% LVV+, as confirmed by adding CK3 to PB and sorting different proportions of GFP+ and GFP− populations (r²=0.9995). The LOD and LOQ of the assay were determined when testing one plate per sample and three plates per sample. With one plate per sample, the LOD is 5.2% LVV+ and the LOQ is 6.4% LVV+. With three plates per sample, the LOD is 1.1% LVV+ and the LOQ is 3% LVV+.

The assay was found to be precise, with an upper limit CV value of 6.8% when sorting one plate per sample. The maximum day to day variability of this assay was 4.4% CV. Consistent with Example 1, % CV was reduced when three plates of single cells were analyzed for each sample, reducing CV 2.1%. Assay controls, acceptance criteria, and plate setup developed in Example 1 were used for this assay.

Feasibility was demonstrated by successfully detecting and measuring % LVV+PB CD45+CD3− cells in 9 samples from clinical trials between 4.5 to 21 months post DP infusion.

The stability of PB samples post blood draw and post density gradient centrifugation was tested. The lymphocyte population in healthy PB post density gradient centrifugation was found to remain stable at 2.9×10³ cells/μL, on average. The sample's monocyte population decreased from 0.4×10³ cells/μL to 0.2×10³ cells/μL, while the granulocyte population increased from 0.1×10³ cells/μL, to 0.3×10³ cells/μL. Though the granulocyte and monocyte concentration varied over three days, the total white blood cell count remained stable. The % LVV+ of PB CD45+CD3− cells from a subject sample remained stable 2 and 3 days post blood draw, but decreased on day 4. The assay can therefore be performed within 3 days of PB collection.

Assay parameter results for PB scPCR were compared to results obtained with CD34+ cells in Example 1 to determine whether peripheral blood components might interfere with the assay (Table 23). PB scPCR assay demonstrated increased linearity, intra-assay, and day-to-day precision, although the false positive rate was slightly higher (0.80% vs 0.17%). The maximum observed % CV for 3 plates per sample was 2.1%, consistent with the statistical margin of error of the assay of ±6%.

TABLE 23
PB scPCR and scPCR (Example 1) comparison of accuracy,
linearity, and precision

| | |
|---|---|
| PB scPCR | CD34+ cell scPCR |
| (this study) | (from Example 1) |

Accuracy

| | | | |
|---|---|---|---|
| False Positive | 0.8% | False Positive | 0.17% |
| Rate | | Rate | |
| False Negative | 4.5% | False Negative | 5.0% |
| Rate | | Rate | |

Linearity

| | | | |
|---|---|---|---|
| R² | 0.9995 | R² | 0.992 |
| Slope | 0.9803 | Slope | 0.949 |
| Y-intercept | −0.29 | Y-intercept | −0.72 |

Precision

| Analyst 1 | Analyst 2 | Analyst 1 | Analyst 2 |
|---|---|---|---|
| % CV of 1 plate/sample | | % CV of 1 plate/sample | |
| 1.7 | 4.0 | 31 | 10 |
| 5.3 | 2.2 | 11 | 10 |
| 5.9 | 6.8 | 14 | 21 |
| % CV of 3 plates/sample | | % CV of 3 plates/sample | |
| 1.2 | 3.0 | 12 | 5.1 |
| Day to Day Variability (% CV) | | Day to Day Variability (% CV) | |
| 4.2 | 4.9 | 19 | 13 |

The assay is suitable to quantify the % LVV+ cells in PB. The assay can be applied to any lentiviral vector containing the PsiGag sequence (LentiD, LentiG, bb2121, LentiGFP, etc.).

### Methods

### Study Design

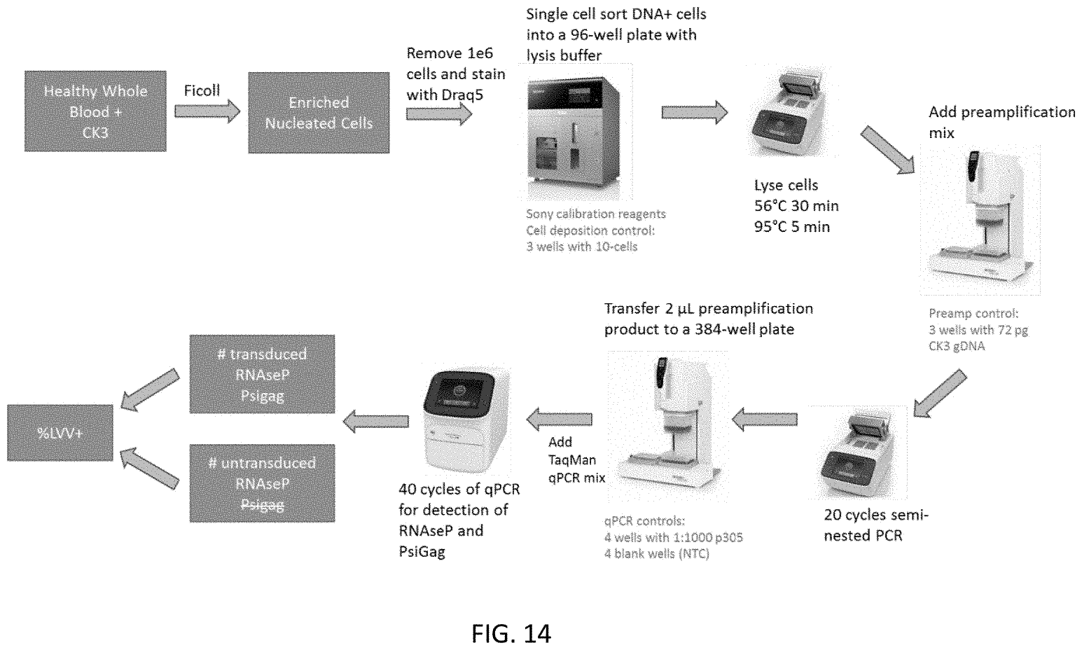

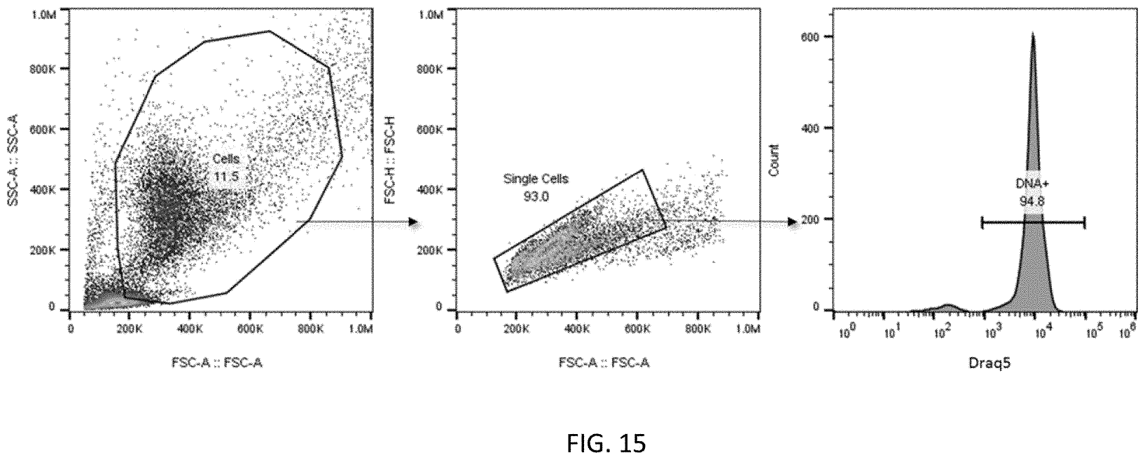

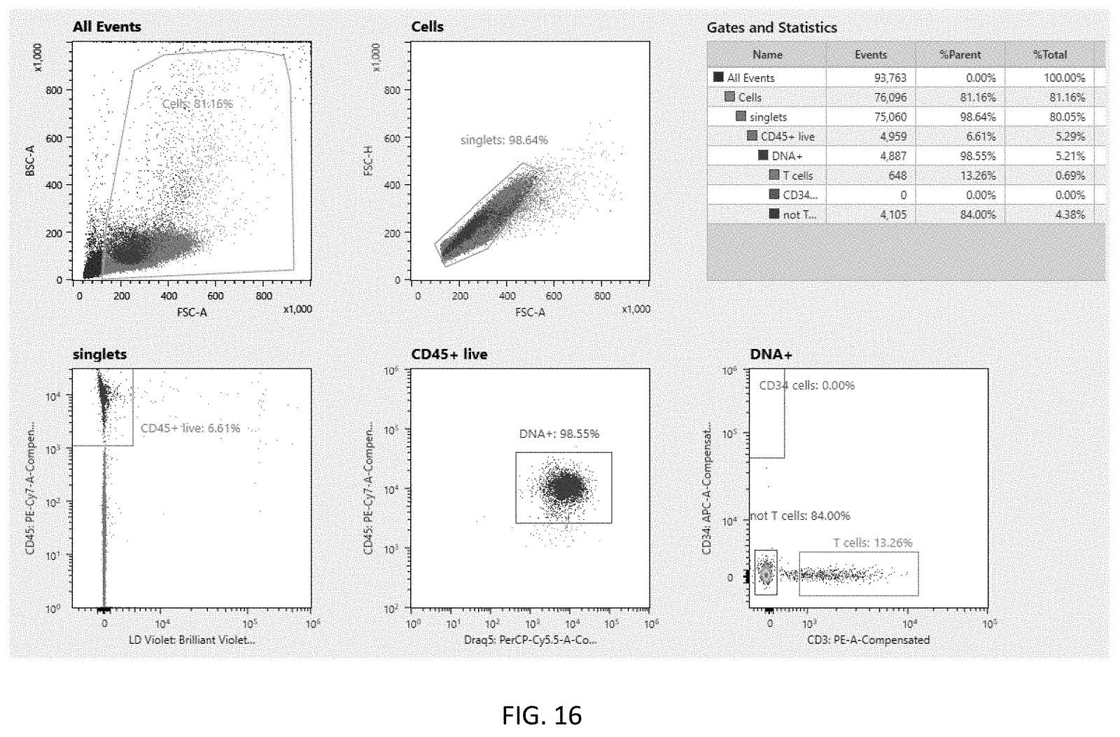

The schematic outline of the PB scPCR assay is shown in FIG. 14. FIGS. 15-16 outline the gating strategy used to sort PB cells for subsequent scPCR analysis. Accuracy was confirmed by spiking healthy donor PB samples with CK3 cells and comparing the percentage of cells that are PsiGag+ by scPCR to the percentage of cells that are GFP+ by FACS. % LVV+ was compared for sorted CD45+CD34− PBMCs and CD45+CD34+ CK3 cells. Linearity was demonstrated by analyzing samples with varying percentages of untransduced (GFP−) cells, and measuring the % LVV+ of DNA+ cells by scPCR. Assay precision was evaluated by spiking HPD16020-Comp1 cells (LentiGlobin transduced CD34+ cells) into PBMCs and analyzing the sample in triplicate on three separate days with two operators.

### Preparation of CK3 Cells

CK3 cells were collected from six confluent wells of a 6-well plate and placed in a 50 mL conical. The cells were centrifuged at 500×g for 5 minutes, and the supernatant was aspirated from the cell pellet. The pellet was resuspended in 1 mL of CK3 growth media (RPMI+20% FBS) and 20 μL of the cell suspension was transferred to a 96-well plate, diluted 1:1 with trypan blue, and counted using an automated cell counter.

### PB Ficoll

The PB sample was removed from 4° C., resuspended, and transferred to a 50 mL conical. 100 μL of sample was placed in a FACS tube to obtain a complete blood count (CBC). For spiked samples, an equal number of CK3 cells was added to the PB. Nucleated cells were isolated from the CK3 spiked PB sample and stored in RPMI at 4° C. until needed for staining and sorting. Minimum PB volume successfully subjected to density gradient centrifugation using ficoll was 0.8 mL, yielding 4×10⁶ cells, sufficient for multiple rounds of scPCR.

### Preparation of HPD16020-Comp1 Spiked Into PBMCs

HPD16020-Comp1 frozen aliquots and 17800556 PBMC frozen aliquots were thawed and counted using an automated cell counter. 7e5 HPD16020-Comp1 cells were mixed with 7e5 PBMC cells. Cells were washed in 1 mL of FACS buffer and the pellet was immediately stained and FACS sorted for scPCR. New aliquots of cells were thawed and mixed each day by each analyst to determine assay precision.

### Single Cell FACS Sorting of DNA+ Cells

Using the post-ficoll CBC, approximately 1×10⁶ cells were transferred to a 1.7 mL tube and centrifuged at 500×g for 5 minutes to obtain a cell pellet. The supernatant was carefully aspirated, and the cell pellet was resuspended in 700 μL of 1:2500 Draq5 in FACS buffer (2% HABS+PBS). The cells were incubated at room temperature for 15 minutes in the dark and then centrifuged at 500×g for 5 minutes. The supernatant was aspirated and the pellet was resuspended in 500-700 μL FACS buffer. The cell suspension was passed through a cell strainer into a FACS tube. If CK3 were spiked in, cells were separated from debris using a forward scatter (FSC)-Area/side scatter (SSC)-Area gate, gated on singlets with a FSC-Area/FSC-Height gate, and gated on DNA+/GFP+ cells with a PerCyp5.5/FITC-Area gate (FIG. 15). If CK3 were not spiked in, the same FSC-Area/SSC-Area gate and FSC-Area/FSC-Height gates would be used, but DNA+ cells were sorted off a histogram of PerCyp5.5-Area (FIG. 15). Nucleated single cells were sorted into a 96-well PCR plate containing 10 μL of lysis buffer (Water, lx Taq Buffer with KCl, 0.1 mg/mL Proteinase K) per well. Plates were sealed with film, vortexed and centrifuged.

### Single Cell FACS Sorting of CD45+CD34− and CD45+CD34+ Cells

Approximately 0.5-2×10⁶ cells were washed with FACS buffer and resuspended in 100 μL of the following antibody cocktail:

| Antibody | Dilution |
|---|---|
| CD3-PE | 1:20 |
| CD34-APC | 1:5 |
| CD45-PECy7 | 1:20 |
| Draq5 | 1:2500 |

Cells were incubated at 4 C or on ice in the dark for 15-30 minutes. Cells were washed in 1 mL of FACS buffer and resuspended in 1 mL of 1:1000 Sytox Blue in FACS buffer. The cell suspension was passed through a strainer into a FACS tube. Cells were gated as shown in FIG. 16, and single cells from desired populations were sorted into wells containing 10 μL of lysis buffer. Plates were sealed with film, vortexed and centrifuged.

### Primers and Probes

PsiGag-F (universal forward primer) 5′-ggagctagaacgattcgcagtta-3′ (SEQ ID NO: 1);

PsiGag-NR (nested reverse primer) 5′-cagctgctgcttgctgtgc-3′ (SEQ ID NO: 2);

PsiGag-R (qPCR reverse primer) 5′-ggttgtagctgtcccagtatttgtc-3′ (SEQ ID NO: 3);

PsiGag FAM probe: 5′-(FAM)-acagccttctgatgtctctaaaaggccagg-(TAMRA)-3′ (SEQ ID NO: 4);

RNAseP-R (universal reverse primer) 5′-gtggtctgaattgggttatgagg-3′ (SEQ ID NO: 5);

RNAseP-NF (nested forward primer) 5′-ggagggaagctcatcagtgg-3′ (SEQ ID NO: 6);

RNAseP-F (qPCR forward primer) 5′-ggagcttggaacagactcacg-3′ (SEQ ID NO: 7); and

RNAseP VIC Probe: 5′-(MAXN)-acctcacct/ZEN/cagccattgaactcacttcg-(IABkFQ)-3′ (SEQ ID NO: 8).

What is claimed is:

1. A transduction efficiency assay comprising:
a) transducing a population of cells from a sample with a lentiviral vector comprising a polynucleotide encoding a therapeutic gene;
b) culturing the transduced cells for a period of at least three days;
c) assaying the cultured transduced cells using single cell PCR;
d) measuring presence of genomic and viral DNA sequences in the cells in the sample;
e) quantifying number of transduced cells, wherein cells are considered transduced when they include both genomic and viral DNA sequences;
f) quantifying number of untransduced cells, wherein cells are considered untransduced when they include only genomic DNA sequences; and
g) calculating efficiency of lentiviral vector transduction (percentage of transduced cells), wherein the efficiency of the transduction is measured as:
$$Transduction efficiency ( \% ) = \frac{\sum transduced cells}{\sum transduced and untransduced cells} \times 100.$$

2. The assay of claim 1, wherein the cells are peripheral blood mononuclear cells.

3. The assay of claim 1 or claim 2, wherein the cells are PBMCs isolated from a subject that has cancer.

4. The assay of claim 3, wherein the cancer is multiple myeloma.

5. The assay of claim 3 or claim 4, wherein the lentiviral vector comprises an engineered antigen receptor.

6. The assay of claim 5, wherein the engineered antigen receptor is selected from the group consisting of: an engineered αβ-TCR, an engineered δγ-TCR, a chimeric antigen receptor (CAR), and a dimerizing agent regulated immunoreceptor complex (DARIC).

7. The assay of claim 5 or claim 6, wherein the engineered antigen receptor is an anti-BCMA CAR.

8. The assay of claim 1, wherein the cells are hematopoietic stem or progenitor cells.

9. The assay of claim 8, further comprising obtaining the hematopoietic stem or progenitor cells from a subject that has sickle cell disease or β-thalassemia.

10. The assay of claim 8 or claim 9, wherein the hematopoietic stem or progenitor cells comprise CD34+ cells.

11. The assay of any one of claims 8 to 10, wherein the hematopoietic stem or progenitor cells comprise CD133⁺ cells.

12. The assay of any one of claims 8 to 11, wherein the hematopoietic stem or progenitor cells comprise CD34⁺CD38^{Lo}CD90⁺CD45RA⁻ cells.

13. The assay of any one of claims 8 to 12, wherein the hematopoietic stem or progenitor cells comprise a pair of β-globin alleles selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, and β⁺/β⁺.

14. The assay of any one of claims 8 to 13, wherein the polynucleotide encodes a globin selected from the group consisting of a human β-globin, a human δ-globin, an anti-sickling globin, a human γ-globin, a human β^{A-T87Q}-globin, a human β^{A-G16D/E22A/T87Q}-globin, and a human β^{A-T87Q/K95E/K120E}-globin protein.

15. The assay of any one of claims 8 to 14, wherein the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, a G9 vector, a BCL11A shmir vector, or a derivative thereof.

16. The assay of any one of claims 1 to 15, wherein the culturing of the transduced cells occurs for a period of 3 to 10 days.

17. The assay of any one of claims 1 to 16, wherein a cell is considered as transduced when it is measured as having a Threshold Cycle (CO value of ≤32 for both genomic and viral DNA sequences.

18. The assay of any one of claims 1 to 17, wherein the viral DNA sequences is a lentiviral vector psi-gag DNA sequence.

19. The assay of any one of claims 1 to 18, wherein the genomic DNA sequence is a RNAseP DNA sequence.

20. A transduction efficiency assay comprising:
a) obtaining a peripheral blood or bone marrow sample from a subject;
b) isolating nucleated cells from the peripheral blood by density gradient centrifugation;
c) assaying the isolated cells using single cell PCR;
d) measuring presence of genomic and lentiviral vector DNA sequences in the cells in the sample;
e) quantifying number of transduced cells, wherein cells are considered transduced when they include both genomic and lentiviral vector DNA sequences;
f) quantifying number of untransduced cells, wherein cells are considered untransduced when they include only genomic DNA sequences; and
g) calculating efficiency of lentiviral vector transduction, wherein the efficiency of the transduction is measured as:
$$Transduction efficiency ( \% ) = \frac{\sum transduced cells}{\sum transduced and untransduced cells} \times 100.$$

21. The assay of claim 20, wherein the nucleated cells are peripheral blood mononuclear cells.

22. The assay of claim 20, wherein the nucleated cells are hematopoietic stem or progenitor cells.

23. The assay of claim 22, wherein the hematopoietic stem or progenitor cells comprise CD34+ cells.

24. The assay of claim 22 or claim 23, wherein the hematopoietic stem or progenitor cells comprise CD133⁺ cells.

25. The assay of any one of claims 22 to 24, wherein the hematopoietic stem or progenitor cells comprise CD34⁺CD38^{Lo}CD90⁺CD45RA⁻ cells.

26. The assay of any one of claims 22 to 25, wherein the hematopoietic stem or progenitor cells comprise a pair of β-globin alleles selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, and β⁺/β⁺.

27. The assay of any one of claims 20, and 22 to 26, wherein the peripheral blood is obtained from a subject treated with a drug product comprising a lentiviral vector comprising a polynucleotide encoding a globin.

28. The assay of claim 27, wherein the globin is a human β-globin, a human δ-globin, an anti-sickling globin, a human γ-globin, a human β^{A-T87Q}-globin, a human β^{A-G16D/E22A/T87Q}-globin, or a human β^{A-T87Q/K95E/K120E}-globin protein.

29. The assay of claim 27 or claim 28, wherein the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, a G9 vector, a BCL11A shmir vector, or a derivative thereof.

30. The assay of claim 20 or claim 21, wherein the nucleated cells are PBMCs isolated from a subject that has cancer.

31. The assay of claim 30, wherein the cancer is multiple myeloma.

32. The assay of claim 30 or claim 31, wherein the lentiviral vector comprises an engineered antigen receptor.

33. The assay of claim 32, wherein the engineered antigen receptor is selected from the group consisting of: an engineered αβ-TCR, and engineered δγ-TCR, a chimeric antigen receptor (CAR), or a dimerizing agent regulated immunoreceptor complex (DARIC).

34. The assay of claim 32 or claim 33, wherein the engineered antigen receptor is an anti-BCMA CAR.

35. The assay of any one of claims 20 to 34, wherein a cell is considered as transduced when it is measured as having a Threshold Cycle (Cₜ) value of ≤32 for both genomic and lentiviral vector DNA sequences.

36. The assay of any one of claims 20 to 35, wherein the lentiviral vector DNA sequences is a lentiviral vector psi-gag DNA sequence.

37. The assay of any one of claims 20 to 36, wherein the genomic DNA sequence is a RNAseP DNA sequence.

38. The assay of any one of claims 20 to 37, wherein the subject has sickle cell disease or β-thalassemia.

39. A method for measuring transduction efficiency comprising:
a. assaying a population of cells using single cell PCR, wherein the population of cells are transduced with a lentiviral vector comprising a polynucleotide encoding a therapeutic gene;
b. measuring presence of genomic and viral DNA sequences in the cells;
c. quantifying number of transduced cells, wherein cells are considered transduced when they include both genomic and viral DNA sequences;
d. quantifying number of untransduced cells, wherein cells are considered untransduced when they include only genomic DNA sequences; and
e. calculating efficiency of the lentiviral vector transduction, wherein the efficiency of the transduction is measured as:
$$Transduction efficiency ( \% ) = \frac{\sum transduced cells}{\sum transduced and untransduced cells} \times 100.$$

40. The method of claim 39, wherein the cells are peripheral blood mononuclear cells.

41. The method of claim 39, wherein the cells are hematopoietic stem or progenitor cells.

42. The method of claim 41, further comprising obtaining the hematopoietic stem or progenitor cells from a subject that has sickle cell disease or β-thalassemia.

43. The method of claim 41 or 42, wherein the hematopoietic stem or progenitor cells comprise CD34+ cells.

44. The method of any one of claims 41 to 43, wherein the hematopoietic stem or progenitor cells comprise CD133⁺ cells.

45. The method of any one of claims 41 to 44, wherein the hematopoietic stem or progenitor cells comprise CD34⁺CD38^{Lo}CD90⁺CD45RA⁻ cells.

46. The method of any one of claims 41 to 45, wherein the hematopoietic stem or progenitor cells comprise a pair of β-globin alleles selected from the group consisting of: β^{E}/β⁰, β^{C}/β⁰, β⁰/β⁰, β^{C}/β^{C}, β^{E}/β^{E}, β^{E}/β⁺, β^{C}/β^{E}, β^{C}/β⁺, β⁰/β⁺, and β⁺/β⁺.

47. The assay of any one of claims 39, and 41 to 45, wherein the cells are isolated from peripheral blood obtained from a subject treated with a drug product comprising a lentiviral vector comprising a polynucleotide encoding a globin.

48. The method of any one of claim 47, wherein the globin is a human β-globin, a human δ-globin, an anti-sickling globin, a human γ-globin, a human β^{A-T87Q}-globin, a human β^{A-G16D/E22A/T87Q}-globin, or a human β^{A-T87Q/K95E/K120E}-globin protein.

49. The method of any one of claim 47 or 48, wherein the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, a G9 vector, a BCL11A shmir vector, or a derivative thereof.

50. The method of claim 39 or claim 40, wherein the cells are PBMCs isolated from a subject that has cancer.

51. The method of claim 50, wherein the cancer is multiple myeloma.

52. The method of claim 50 or claim 51, wherein the lentiviral vector comprises an engineered antigen receptor.

53. The method of claim 52, wherein the engineered antigen receptor is selected from the group consisting of: an engineered αβ-TCR, and engineered δγ-TCR, a chimeric antigen receptor (CAR), and a dimerizing agent regulated immunoreceptor complex (DARIC).

54. The method of claim 52 or claim 53, wherein the engineered antigen receptor is an anti-BCMA CAR.

55. The method of any one of claims 39 to 54, wherein a cell is considered as transduced when it is measured as having a Threshold Cycle (Cₜ) value of ≤32 for both genomic and viral DNA sequences.

56. The method of any one of claims 39 to 55, wherein the viral DNA sequences is a lentiviral vector psi-gag DNA sequence.

57. The method of any one of claims 39 to 56, wherein the genomic DNA sequence is a RNAse P DNA sequence.
